SEQUENCE LISTING (14 sequences)

SEQ ID NO: 1 - DNA, 6995 bp, Cucumis sativus
  misc_feature 1..6995: note = Wild type Genomic DNA sequence CsYLS9
  source 1..6995: mol_type = genomic DNA
cataaaatcgacaaaaccaataaaaataatttgaaaacgtgactcgcaggttttgataattctttaaagaaataaagataaataaatgagacaaaaatttaaataaggtgtctaaaaccaaaatactgaaaaactgatacatagaaacggaaacaaaattgagtctccatatgacatgtcacagacccttattgtcacttggctttccagatcctctacctctgcctgaaagattaaatacagaaaagagtgagtatataaaatatatccagtaagggatcatctactggtctcgctaggtgatttgttaactttccattagaaacataataatagtgttgtgtgttcaatggagcacacctaggcgagtgagatactacgaacacacctaatcgtgtgagcaatctcgtaggaacacccttagtcgtgcgagtgatcgtagatacacactgagtgagaccatatgaacacccctagtcatgtgagtgatcgtacatacacactcttaaacatgtctgtgatatgtaggtacacctcttattgtacacagtctaaacgatctcttagtaataatcttgtagcaatgatctcgtagcaagtctaaacgatctcatagcaagttgaaacgatcttgtaccctcgtacctagccttaaacaatcttgtacccagtctaaacgatcttatacctagtctaaacgatctttgtatctagtttaaacaatcttgtaccaaatcaaaacatctattagtgatagtggtctatctgtctatctaggataaacaactgatcgtttagacattggtacacgatcgtttagatcttgtaccaaaaagaaaaaaaagaagatgagaaatgaagaaaaaggaagaaattctggaagaagaaataaaaaaattggtaaatatttacataaataaccataatattttaaaatgaataagaagtaataatacaaagaaattaataataagaaaaagagacgaataaatcgcaaaaaagaaaaagaaaaagaagtgaaaaatcgtcagcactattaagggtaaatttggaatttatgaaagtaccatgagttttttaattttattatatgggtcgtaaatattttggtcatgttttgttatatttataaaaattaaccaaacataatagtacagaaaattatacatggtgcagtaaaataacctacatttaatttaggaaaattgtattaaatgacaaaaatatttagaaaaaaacagctcatgacacttattttttgtatattgtgaatatgacaaaattagtgatatcagatgactattagatgataatcatagggccatcggagggctattgacgataatcatagagttatcgacttttaaatttgctactctcgcaatttagaaaatgtaatgacatgagccttattgtcataatttggtggttccagaaaattgattggaaaagaggagtaggaaatgatgagatcaagaaccaaacaagtaagccatagaaagttctccacttgctctggtggttccatatgcaagtgggaaactttctatggcttactcggtcttgtcattttctgctcctcttttctcaatcaattttctggaaccactagagcaaactagagttgtttctgtttgagacaacctcatatttctttcgacgcaactcgtaataatgttcaactttcctctttgcacatgctgctcatgtccctatgatcatactaggcaattatgctgaggcaatgcttttagttattcatgtaatctttgaacctacaaacgtgtttttttttttaatatttgacttaattttaatataattttgatttgatattttaataaattttgaataaattttatgttaattgtttttagaatttgattaatttaaattgaattcgatcccaatagtgaataaaattaaataataatatattaaaaaattattacaaaaacgttttcctgatgcacaaagcacgttggcatggacacaattgctgacgctttgaccagatgttagacgagatttttctcgatgtctcacgtgagtcagcgaaccccaatcgggcaaacgtttttgtcgacgtcatcgtgtttgctgacatctcgacgctgtaatggtgacgttcttgtcgacgttagttttggcgtcgggatagatattctcgacgtgtttttgtgaatttactgacacatgtgtgcgtcagatgaacccttacttcttgtagtgataatgttcttttctattttaatttggttgaaaaaatacattttccataattcttaaaaaagaatgtctctccattttcaaaatcttgatgttcggttgatccacaaggtttccattacaagaaattgtgtttttagcggcgcaccaaaaatgtcactaaaatgcaaaatacatcgctaaaagtattagcgacgcaaagccatttgtcgctaaatccatgtcgttaaaagttttagcaataatgcaacaattactcaccgcaaatgagatacttttagtgacgttttcgtgttacttttagtaacataatttagtaataaataaatgttgttgttaaaggttaatttgaaatcattaattacaaatatcttttgcgacatattgcagtgcgtcactagtaatgcttttgtgacgtaattgaaaatgccaataaaaatattttattatataaaaaaataatatccaatttattatataaatctgttataaaatgttaaatataataatatgaaaaactatcacgagagaatgtgcaacaaaaccaactttatctcaaaaagaaatatatgtaaagaaacttcaataaattcataaatagttcaagctttcactgatgtattaaggatttttcatatgcaacttgtttgtttttctctttttgccacatgggctttttatgtataaacttgaacattattttagccattacccacatctaaacaaaataaagcgtacttaactcaaatcttgaactacaagtaaacacaaaatacaccctaattttaaatagtgaggatatatacctgtaccccttcattaagcaaaccaaaatcatataatatcccttcacttatacatgagagaacaaagatcaaaaccaaaaaagaatatatatatatatatatctatttttgaagaacatgaggagtactactacacagggagaaggagcatcatcctccattattgaggcaccaaaacgaagcttctgtagacaacgtgagacaacaaaacgcacaagaatcataagaatcataggaagaagtttgttgtctgtaataatcttcttgagtgttgcaattatcacatgttggcttgttgttttccccagaaccccacgtctcatggtggaaactagcaaagtgacagcccatggttcaactaatagacacctcaatgcaaccatagttttctacatcaaaagctacaaccctaacaaaaaagcctccattcacatggattctgtgaagatgatagtcagtgattatatggggctaccgtttcactccaccatccccaccttcacgttgatgcctcgaaacgagatggtcttcaactcaaccgttcgtgtcaacttcatgtacccatttgggcgcccggtgcattcggactgggtacatctagagcttcgcttctctgctcaagttaggtacgtacaaacatgtatgaacatagaagtgttgttggttttgttggtgtaaattatttatttttattcttactttagaattagaattagttttgaaaaactcttaaaattttaggagtattattttttacacaaaatggtaactgttctatcatttttgaattctttttgacatataagagtattttttaaacatttgaaagttgaaatatattttttgacataagtaaaaagttggaagagatattttgtataattttgccttaattgcttttagattttaattaaaattaaaccgatggatactactaataccaaaatttgcctatatctttaataagtcatggctttgtttgtatcagttacattgtgaacagatggaggtcgaaaccacgattgctggagatctattgtgatcacctttggcttaggattaatgattctacccctaattttgataaaaccaaatgcagagtggatctttaaagagatttgatctagatgtgttgttgatcttgtttaagaagatatgttattcatgctggttatataattccaaaagatttgtaattgaaatattcatttcctttaggtttaatttctttctttatcaatagagttcttaatgttttataatgtttttttttaatggttaaaatatcattttcatcaatgtactttgataattgttctatttagatctatatatatatttgtgctttaattcaaaatgtcaaattttagtgcctataaattggatataaaaatcaaacgtgttggcaaaggaaaacaaaagtgatataggctatagaaaagtctaacacatctctggctttagaaaccatttttttcctatcctttcctttcttcttttctcctctccaacttcatcttcactgtggttatcgatcattctctccctttctcttctttctctattttccctctttatcccttcgacgcttgtcgatgttcacggccaccatcttgtcgccgtccatagctcatccattttgatgaacccaaattagagctcaataaatttaaagtatgtttgatcaaaaccgattcgaaatatcatgtgatgtgtaatatacaaatgaatagaaggtaaaggaattcaacgttttgcaaacaaacacccaaatctattattccatatgatctggcttttggttcctgtagttgtggcccaataattagtagcgcgatgttaagctatggacattggcggtgatagagcgcatatgtggtctatagatgggagcagttgaacaacaatgcgaccgtgagcaatagtaagtattacattggtgttgtagcgacccgacttcttatgattaccaacaaattagatattgttatatggataatatcataaagaaattagaaaatattaacgtagaacacgatatatctattagagtcggtctaatgaattcacgttttctcttttaagacaagtttattcattctagggccggaatgagccggagtttcatgagtaattgtctcttaagatgaaataggattaactttctcatgtaacaacacctcgtatacaaaatccaccgaactatactttgtaaaaataaaccgaacttgaacaaagtttacaaaagagagaaatgtctatgagagagaatataattatgaaaaaaattctcttaaaagcaacttgaaggagtggcttatttaatttatagactattcgtgatcattcgaatagtcacatttctttttcaaattgttgtttttgaaaaaacatatttattcatgaaacaatataacgctgtatacgaatagccatataccatttatttcaatagttgtgcatgcaaaatttttaaagcagatcatccgacgaattttagccacaaaatgaaattaattccacaaggtaaatatcattcatgaagagaggattttaagggcttatttcatagagaaatctagactatattttatgttttcagatttattgagttcaacaaatactacaaaaaattttggtctcagccaacaattttaattttctgataacgtaaaaaaatgtgaacaaagatatgacgtgccaacgcaattccattacgtggacgcataatcgtttttgtcgaccaaaatatatttgtgggtaaaaatgttgcgcagtcttcaaaaacttttttaagttggatttctattgacatttattttagtttatgaccatattttaaatatttgccgacataatttacgtgcgaaaaaccccacttttaaaaaaacctattctctttatttattttagcacactttctaaacaacccacattttcccccttctaaaaacccacataatttgtccctctttttaagttattaggttaggcgattaggttccttctgcttcaaaatcgcctgacgtctctccatttgatccatcattatctccatccctcgccaagaataactctcttcctgcagttgttaactctcaacctcgctcacgaaccattgtcggtctccaatcgtcattcaccgttcccgtcgtcgagcctctttcaccgcccgtccaacgtagatcaaagttgccattgctgttggtgagtttctcgtgaataatctctctcttttacccgcttttcccccttattttaaatttcagtttatgttcatatgcaaacttttttgtttcctgcattaatgcaattttcccttcagttggatacataagtctttttttttacactttatgggtacaaatgtaatttcaattatttattttgatacttgattcatttgaaacaacagtagttaagctcttattcatcttaatgtatctttttcaggtttgcctatgatgactctatttttgtgaagattttctaaacgggctaactttttttttttgtgtgtatttaatatttttttgttaccaaaataaaaattaatatagctcatatgactatacacactaaagaattatgcttggttaaattttgaccaaaatattaatgatatataatattaataataaatgtataagaaattaaaataatttttgtcatgctttttgtgctactggaaatatgttggtagttagaaagctcatattgcagttttgtgcgttggcaaaaaaaaagacaaatattatatgccaatgcatcatttgtggccaacaaacaaatttctatccaacaaaattatatgacacatgtatgtatgtatatatatatatatatatttctgcttacgagtttagtgtgggaagaagttttctttttgcagctgacaaaaatatttttgttggtagaagcaatttcagctgacgaaaaaaaatgtaaatatataaattc

SEQ ID NO: 2 - DNA, 633 bp, Cucumis sativus
  misc_feature 1..633: note = Wild type CDS CsYLS9
  source 1..633: mol_type = genomic DNA
atgaggagtactactacacagggagaaggagcatcatcctccattattgaggcaccaaaacgaagcttctgtagacaacgtgagacaacaaaacgcacaagaatcataagaatcataggaagaagtttgttgtctgtaataatcttcttgagtgttgcaattatcacatgttggcttgttgttttccccagaaccccacgtctcatggtggaaactagcaaagtgacagcccatggttcaactaatagacacctcaatgcaaccatagttttctacatcaaaagctacaaccctaacaaaaaagcctccattcacatggattctgtgaagatgatagtcagtgattatatggggctaccgtttcactccaccatccccaccttcacgttgatgcctcgaaacgagatggtcttcaactcaaccgttcgtgtcaacttcatgtacccatttgggcgcccggtgcattcggactgggtacatctagagcttcgcttctctgctcaagttagttacattgtgaacagatggaggtcgaaaccacgattgctggagatctattgtgatcacctttggcttaggattaatgattctacccctaattttgataaaaccaaatgcagagtggatctttaa

SEQ ID NO: 3 - protein, 210 aa, Cucumis sativus
  REGION 1..210: note = Wild type Protein CsYLS9
MRSTTTQGEGASSSIIEAPKRSFCRQRETTKRTRIIRIIGRSLLSVIIFLSVAIITCWLVVFPRTPRLMVETSKVTAHGSTNRHLNATIVFYIKSYNPNKKASIHMDSVKMIVSDYMGLPFHSTIPTFTLMPRNEMVFNSTVRVNFMYPFGRPVHSDWVHLELRFSAQVSYIVNRWRSKPRLLEIYCDHLWLRINDSTPNFDKTKCRVDL

SEQ ID NO: 4 - DNA, 642 bp, Cucumis sativus
  misc_feature 1..642: note = Modified CDS CsYLS9
  source 1..642: mol_type = genomic DNA
atgaggagtactactacacaaggagaaggagcatcatcctccattattgaggcaccaaaacgaagcttctgtagaaaacgtgagacaacaaaacgcacaagaatcataagaatcataggaagaagtttgttgtctgtaataatcttcttgagtgttgcaattatcacatgttggctggttgttttccccagaaccccacgtctcatggtggaaactagcaaagtgacagcccatggttcaactaatagacacctcaatgcaaccatagttttctatatcaaaagctacaaccctaacaaaaaagcctccattcacatggattctgtgaagatgatagtcagtgattatatggggctaccatttcactccaccatccccaccttcacgttgatgcctcgaaatgagatggtcttcaactcaaccgttcgtgtcaacttcatgtacccatttgggcgcccggtgcactcggactgggtacatctagagcttcgcttctctgctcaagttagttacattgtgaacagatggaggtcgaaaccacgattgctgggatctattgtgatcacctttggcttaggattaatgattctacccctaattttgataaaaccaaatgcagagtggatctttaaagagatttga

SEQ ID NO: 5 - protein, 213 aa, Cucumis sativus
  REGION 1..213: note = Modified Protein CsYLS9
MRSTTTQGEGASSSIIEAPKRSFCRKRETTKRTRIIRIIGRSLLSVIIFLSVAIITCWLVVFPRTPRLMVETSKVTAHGSTNRHLNATIVFYIKSYNPNKKASIHMDSVKMIVSDYMGLPFHSTIPTFTLMPRNEMVFNSTVRVNFMYPFGRPVHSDWVHLELRFSAQVSYIVNRWRSKPRLLGSIVITFGLGLMILPLILIKPNAEWIFKEI

SEQ ID NO: 6 - DNA, 6110 bp, Cucumis sativus
  misc_feature 1..6110: note = Wild type Genomic DNA sequence CsHSFA2
  source 1..6110: mol_type = genomic DNA
ctcaaggtaagctcggtgtcattcttcgacgatgaaaagacagaaaccgctacctcccgttggtcccgggctaggacgagagctgcaaaggtaaattcttggtattagtatgcttcattagttgtggtttagaacaataatgatgttctctaacttgttcagcttgggaaaggtttatcaaaggatgagaatgctcaaaaattagctcttcaacactggcttgaagcagtaagttcatatagttcaaacttttagaggttagagaagaggatgttgaccgttttattgaacttcttttggccagattgatccccgccaccgctatggacataacttgcatttttactatgatgtttggtttgatagcaagagcacacaacctttcttctactggtaatttccccaatcacattgctggtatcattacaccgaaagctttcattcttatccttcaagcttccttttaaaaggttggatattggtgatgggaagagggttaatcttgagaagtgccgtagatcagttctgtataagcaatgcatcaagtaccttggaccggtaagtcgcctgattagattgtagtttctactccattacgagcagtaagcaattgtttctattcatttctgttgcagaaagaaagggaggagtacctggtgattgtggagaatgggaggcttgtttacaagcaaagcagaatacccatcaccacagttgaagattccaagtggatttttgtactcagcacgtcaagagatctgtatgtgggacagaagaagaaaggtcgctttcaacactccagcttcctgtctggaggagctataacggctgcaggaagattagttgccattgatggaattctcaaggtgcataattgaatgcttgagcatatgagtgttttgtaggataatcattctgtagagcgtgtaattcatagaggatgatgttttcatttgaattttctttaggctatatggccatacagtggtcattacctcccaacagagaacaattttaaggagttcattagtttccttgaagaacatacagtggacttgaccaacgttaaggtaagaaattagatatataactacatggatgtagttttctatatcagcttcttttcagattgcacaacttgttttcttgaattttctcaatggggattcctgcgttattatacagagatgttcggtagatgatgacaactactcacttaacaacacgagcgaggaaacaacggaaacaacttcagaagacatggttgcagatgatgttgatttggcagtacccgtcaagttggtcacaactaatgagcggcaagaggatcagggtagcagcagagaggcaccgttgatagacataccaaagcgcttgctgtgcagatggagcagtggagttggccctagaatagggtgtgtgaaggagtatccagcagagttgcaagcacgagcactggagcaagtgaacttgtcaccaagaccgtcaccaggtttcttcggaggctcgcttccaataccttcaccacggccgagcccaaaaatcaggatgtctcctaggctctcatatatggggatccccagccccagggtgcctgtgtgtctgacagcgcccatctaaagcttctgcctcttgtgcccctaacaaaacctctgatttgtcgcattttcatgtacatggaactcatccttctttctgcctggaatgattcgagtagtacatatcttgtttcactggatcattgacttccctggaataaagaaccaatattttatttaccaatcccctaaatgcgagggaaaaggaaaacaattctatagttcaggggcccatgttattttggctgcgtgttcaatcccagcaaagcaataaatgcaagagatctcaaatgtgtgcctccggagcccttccatacaaaataacttgtgagatgctttttcatgccatggaaaattgcaaacaaaccatgaacttgaaaaaccccatccactgacattgaccaaaacctctcgccctgtaaggagcattctaaagtgagaaagagagagcagctacctctaggtatattaatttcctttctcttgagaaaacaaccgtgcggattttatcttcttctgccaccaaaggatatccatcaaccaacagtacagaaaatcataaaagaattctactcagttaatgaagcctgaatttctaaacttgtgtttgttccattttacatagctgagcttaacatcattaagctcaaacaagaactatcaaccaaggaccaatagcctttcaagatctaatacaattttatcaggaatctattccctgagttatgatatagctgcgttttgttatcctccacaaaaagtagtgagcatcaaacatactttaagggaaaagatttggcagactcgtacatttcaaatgaataacagttattgggttccaattaatgaatggaataattagtcttacgttaatggtaaagaaataagaatatgactgaatcttctcgcaggtttcctttctctgaacatgcctgcaaataaaaccatggatgaacaaaaaaaaaaaccacaaaccaattgaacacaaccaagaatattaagtacataatattgaaaaagccaatgagtttcaaaggatataaggaaacaggaacaatctgtggctcccctgcaacatccacggttccttcctacacgagccagctgaaacctattccacacgaagagaagcaaaatggcaaacatattaactacactgttatatctatggctttgaccggagaaaccccatttgatcaacgagttcctgcaagtcctcagtccaatctaggggttcagcaatcagatcctccacttccacatcaatgtctgattgattaacaatgattgtgggttcttctggatgtccagccacaagatcttcaacccaaaggtgactgaaaatccccgattcttcgtggacagaatgacccaagtcggaaacagggtccgcgatctcaatgcttgactcgtcttcaaagttgactgttaagagcgtctctatatctggttcagaagtttctagctcttcttgtttaagagctacaggtacattctcatccaggagattctctacacttgggctggcagttagtcttctttttcttccaatttctacacctctcaattcccttccctgattactattaataaatttctgaatgaatgaaggattcttgagggctttgctgaggaatgtcatgatctgcttctgtttactctcagctttctctaacctatcctccatggtcattattttatctcttgagctctggtgttgctgcctcaatctcactaattccgccattaacgtgcttctgtcccttctcaacctctcaaggtcagcttccagtccaaattgccctaattcaacacatgttcctccatggtgctgaatgctttgttgtgaatgtcttctcctctttatggttcttagcagattccgttgtcctcccagaaacccctcattcgcaaactcccatcgatcaggatcgactttacgaaaacccttaattgaagaatttcaacatcaacgagattaggttatgaaattttcaaacatacttcagaaacaaaaacccacaacactgtggacgttgtatcaattaaaactccaaactaattcgttgcgttcataattcctacccatttcaattagctccattcaaaatcagtttaaattcacatacgtgaaattatatctctaccaaaatactatcaaaatatgaagaaaatcgatacgtcaacaagtaaagaaaacggagagaaagacaacgaatatcaatttcagtgaaaaacttacataagtattgagttgacgaatgaaactagagaagttggagtgtttgaagtaacgaggcagcaaggtactagagaatttgtgataatcccaaacgatgaaactattacgagctctactccatgaaacaatagagtccgtcaatggatcttcgaccatttcaaaggtcttggtcagaaatggaggtggaccaacgtcgtgtaagccttcaatcggctggggagtaacggatgaagaggaagaagaaaaagaagaagaagacgcggtagcggtggccacaagtgattcctcgggtttcactttcagttcatccattacagatttgatcgtaagcgaagttagtcaaagattgagaagctaatgggcttgattagttgaagaagaaatgagtttagagattgattgattgataaatggatggaagaaactgaattttaatggcgtcggagtggaatgttctaggactcagcaatgtctttttcaggtgtttgtcagaagcttcttcccccttgtccacctcaccaaaaatatctaatcgaaacttctcgtttcttccaggtcttttttttaatatcagtcctttaaaattttcataaacaaaaatatttatgattgcctaacaaaatcaaaatcaagatgttgaggaagtcggacagtttttcaaaaatattttaagtttaccttcctttctatcttttttcttcaccaattttttttttttttcttttctcaatttgttacttgtttgtttctaacgtatcaaaaatggtaaaagctaaacgacaaatatattatgcattgggtgtcaatttattgaaatactttgtatagttggcctctgaactcttttcgttttcaaaattattctacatataaatatcttatctttttgttatgttttaaaaaatattgtttattttataaaattaagatcgttttagtatagtaaccaaaatcacatatttagtctttaaaatttaaaattttgaaatactttgagtgtcaaagtaaaatgatgtttttcattccaacaaaatactcaaatgctaaaagatagatgactaaagattttaatagacatttttatacccatgttttgcacaatattaaaaaatggtccattccttttaacaaaaccttagattgtcttcctttattatcaatatttgaaactataaaataatttgttaggaaataagtaaaaaagaaagtaagaaatgagaaataatgtaatgattatatttgcactaatcaatcaataaaaaaagtttaattttgttaagaagcatttttttaaataacaaaataaattaaaatatcaaaattttgaattatgtcactcatactgttatatcattctaacacattaatagttattcgtgataaaacttgttatagacggtaaatattttataaaatctattgttttctaaaattctctttttcttaattataataacaatcattgtttttagtaaatattaatttattaatcaacttaaacatcatctcaggtataattgaacaaagagctgaggcctccatttgttggattgtaaattgggctctaaaagcccacaaaaaaggattttcctttcatgattgaaatccaagggatggatgaattttaacctaccaaataattgtagtcttcatcctaaactcatctatttatatccattgggtcacttgcaaaaaaaatggaaaaagttaccgtccacatattttattatttacaaaagaatcataaaataaaataattataaaaatatgaatttgaaatttttttatttatatttgcaaaatcacaaacgttacatagaccgctaaattaatttgtcatccaatacaattttccatatccatttacataggaagcttttaatttcatcaaattaatttaaaataatttacccgtgttgttgtagctttttaattaatgtttattgagcaaatcttagaacaatagacatacaatttcatgtcgattcataaattttatatagctagacactatataataaacatgtttttttctttttttttttttgtaaacgtaggacttgaactcttggacacgaccacaacattacaacaacattttgtaaagaataaagcattatttaatatatattataatttgacaactacaaaatattattaaaatataaaaaccaatgaaagggcacatgtcctcactggctcctacataaatttcttagtgctcatgtcgacaaggcacagttcattggatgcaagtagtttactctgca

SEQ ID NO: 7 - DNA, 1095 bp, Cucumis sativus
  misc_feature 1..1095: note = Wild type CDS CsHSFA2
  source 1..1095: mol_type = genomic DNA
atggatgaactgaaagtgaaacccgaggaatcacttgtggccaccgctaccgcgtcttcttcttctttttcttcttcctcttcatccgttactccccagccgattgaaggcttacacgacgttggtccacctccatttctgaccaagacctttgaaatggtcgaagatccattgacggactctattgtttcatggagtagagctcgtaatagtttcatcgtttgggattatcacaaattctctagtaccttgctgcctcgttacttcaaacactccaacttctctagtttcattcgtcaactcaatacttatggttttcgtaaagtcgatcctgatcgatgggagtttgcgaatgaggggtttctgggaggacaacggaatctgctaagaaccataaagaggagaagacattcacaacaaagcattcagcaccatggaggaacatgtgttgaattagggcaatttggactggaagctgaccttgagaggttgagaagggacagaagcacgttaatggcggaattagtgagattgaggcagcaacaccagagctcaagagataaaataatgaccatggaggataggttagagaaagctgagagtaaacagaagcagatcatgacattcctcagcaaagccctcaagaatccttcattcattcagaaatttattaatagtaatcagggaagggaattgagaggtgtagaaattggaagaaaaagaagactaactgccagcccaagtgtagagaatctcctggatgagaatgtacctgtagctcttaaacaagaagagctagaaacttctgaaccagatatagagacgctcttaacagtcaactttgaagacgagtcaagcattgagatcgcggaccctgtttccgacttgggtcattctgtccacgaagaatcggggattttcagtcacctttgggttgaagatcttgtggctggacatccagaagaacccacaatcattgttaatcaatcagacattgatgtggaagtggaggatctgattgctgaacccctagattggactgaggacttgcaggaactcgttgatcaaatggggtttctccggtcaaagccatag

SEQ ID NO: 8 - protein, 364 aa, Cucumis sativus
  REGION 1..364: note = Wild type Protein CsHSFA2
MDELKVKPEESLVATATASSSSFSSSSSSVTPQPIEGLHDVGPPPFLTKTFEMVEDPLTDSIVSWSRARNSFIVWDYHKFSSTLLPRYFKHSNFSSFIRQLNTYGFRKVDPDRWEFANEGFLGGQRNLLRTIKRRRHSQQSIQHHGGTCVELGQFGLEADLERLRRDRSTLMAELVRLRQQHQSSRDKIMTMEDRLEKAESKQKQIMTFLSKALKNPSFIQKFINSNQGRELRGVEIGRKRRLTASPSVENLLDENVPVALKQEELETSEPDIETLLTVNFEDESSIEIADPVSDLGHSVHEESGIFSHLWVEDLVAGHPEEPTIIVNQSDIDVEVEDLIAEPLDWTEDLQELVDQMGFLRSKP

SEQ ID NO: 9 - DNA, 1089 bp, Cucumis sativus
  misc_feature 1..1089: note = Modified CDS CsHSFA2
  source 1..1089: mol_type = genomic DNA
atggatgaactgaaagtgaaacccgaggaatcacttgtggccaccgctaccgcgtcttcttctttttcttcttcctcttcatccgttactccccagccgattgaaggcttacacgacgttggtccacctccatttctgaccaagacctttgaaatggtcgaagatccattgacggactctattgtttcatggagtagagctcgtaatagtttcatcgtttgggattatcacaaattctctagtaccttgctgcctcgttacttcaaacactccaacttctctagtttcattcgtcaactcaatacttatggttttcgtaaagtcgatcctgatcgatgggagtttgcgaatgaggggtttctgggaggacaacggaatctgctaagaaccataaagaggagaagacattcacaacaaagcattcagcaccatggaggaacatgtgttgaattagggcaatttggactggaagctgaccttgagaggttgagaagggacagaagcacgttaatggcggaattagtgagattgaggcagcaacaccagagctcaagagagaaaataatgaccatggaggataggttagagaaagctgagagtaaacagaagcagatcatgacattcctcagcaaagccctcaagaatccttcattcattcagaaatttattaatagtaatcagggaagggaattgagaggtgtagaaattggaagaaaaagaagactaactgccagcccaagtgtagagaatctcctggatgagaatgtacctgtagctcttaaacaagagctagaaacttctgaaccagatatagagacgctcttaacagtcaactttgaagacgagtcaagcattgagatcgcagaccctgtttccgatttgggtcattctgtccacgaagaatcggggattttcagtcacctttgggttgaagatcttgtggctggacatccagaagaacccacaatcattgttaatcaatcagacattgatgtggaagtggaggatctgattgctgaacccctagattggactgaggacttgcaggaactcgttgatcaaatggggtttctccggccaaagccatag

SEQ ID NO: 10 - protein, 362 aa, Cucumis sativus
  REGION 1..362: note = Modified Protein CsHSFA2
MDELKVKPEESLVATATASSSFSSSSSSVTPQPIEGLHDVGPPPFLTKTFEMVEDPLTDSIVSWSRARNSFIVWDYHKFSSTLLPRYFKHSNFSSFIRQLNTYGFRKVDPDRWEFANEGFLGGQRNLLRTIKRRRHSQQSIQHHGGTCVELGQFGLEADLERLRRDRSTLMAELVRLRQQHQSSREKIMTMEDRLEKAESKQKQIMTFLSKALKNPSFIQKFINSNQGRELRGVEIGRKRRLTASPSVENLLDENVPVALKQELETSEPDIETLLTVNFEDESSIEIADPVSDLGHSVHEESGIFSHLWVEDLVAGHPEEPTIIVNQSDIDVEVEDLIAEPLDWTEDLQELVDQMGFLRPKP

SEQ ID NO: 11 - DNA, 528 bp, Cucumis sativus
  misc_feature 1..528: note = Wild type CDS CmYLS9
  source 1..528: mol_type = genomic DNA
atgaggagtacaactacacaagaagaagaagcatcatcctccattgttgaggcaccaaaacgaagcttctacagacaacgtcatgagacaacaaaacgcacaagaatcataagaatcataggaagaagcttgttatgtgtaatcatctttttgagtgttgcaattatcacatgttggcttgttgttttccccagaaccccacgtctcatggtggaaactagcaaagtgacagcccatggttcaactaatagaaagctcaatgcaaccattgttttctatatcaaaagctacaaccctaataaaaaagcctccatttacatggattctatgaagatgatagtcaaggattatatgggcctaccatttcactccgccatccccaccttcacgttgatgcctcgaaacgagacggtcttcaactcaaccgttcgtgtaaacttgatatacccatttgggcgcccggtgcattcggactggatacatctagagcttcgcttctctgctaaagttaggtacgtacaaatatga

SEQ ID NO: 12 - protein, 175 aa, Cucumis sativus
  REGION 1..175: note = Wild type Protein CmYLS9
MRSTTTQEEEASSSIVEAPKRSFYRQRHETTKRTRIIRIIGRSLLCVIIFLSVAIITCWLVVFPRTPRLMVETSKVTAHGSTNRKLNATIVFYIKSYNPNKKASIYMDSMKMIVKDYMGLPFHSAIPTFTLMPRNETVFNSTVRVNLIYPFGRPVHSDWIHLELRFSAKVRYVQI

SEQ ID NO: 13 - DNA, 1104 bp, Cucumis sativus
  misc_feature 1..1104: note = Wild type CDS CmHSFA2
  source 1..1104: mol_type = genomic DNA
atggatgaactgaaagtgaaaccggaggaatcacttgtggccaccgccaccgccactgccaccgcgtcttcttcttcttcttcttcctcttcatccgtcactccccagccgatctcaggcttacacgacgttggtccacctccatttctgacgaagacctttgaaatggtcgaagatccattgacggactctattgtttcctggagtagagctcgtaatagtttcatcgtttgggattatcacaaattctctagtaccttgctgcctcgttacttcaaacactccaatttctctagcttcattcgtcaactcaatacttatggttttcgtaaagtcgatcctgatcgatgggagtttgcgaatgaggggtttctgggcggacagcggaatctgctaagaaccataaagaggagaagacattcacaccaaagcattcagcaccatggaggaacatgtgttgaattagggcaatttggactggaagctgaccttgagaggttgagaagggacagaagcacgttaatggcggaattagtgagattgaggcagcaacaccaaagctcaagagagcaaataatggccatggaggataggttagagaaagcagagagtaaacagaagcagatcatgacgttcctcagcaaagccctcaagaatccttcattcgttcagaaatttattaatagtaatcagggaagggaattgagaggcgtagaaattggaagaaagagaagactaactgccagtccaagtgtagagaatctccaggatgagaatgtaccagtagctgttaaacaagaagagctagaaacttctgaaccagatatagagacgctcttaacagtcaactttgaagacgagtcaagcattgagatcgcggaccctgtttccgacatgggtcattctgtccacgaggaatcggggatcttcagtcaattttgggctgaagattttgtagctgtccatccagaagaacccacaatcgttgttaatcaatcagacattgatgtggaagtggaggatctgattgctgaacccccagattggactgaggacttgcaggaacttgtcgatcaaatggggattctccgatcgaagccgtag

SEQ ID NO: 14 - protein, 367 aa, Cucumis sativus
  REGION 1..367: note = Wild type Protein CmHSFA2
MDELKVKPEESLVATATATATASSSSSSSSSSVTPQPISGLHDVGPPPFLTKTFEMVEDPLTDSIVSWSRARNSFIVWDYHKFSSTLLPRYFKHSNFSSFIRQLNTYGFRKVDPDRWEFANEGFLGGQRNLLRTIKRRRHSHQSIQHHGGTCVELGQFGLEADLERLRRDRSTLMAELVRLRQQHQSSREQIMAMEDRLEKAESKQKQIMTFLSKALKNPSFVQKFINSNQGRELRGVEIGRKRRLTASPSVENLQDENVPVAVKQEELETSEPDIETLLTVNFEDESSIEIADPVSDMGHSVHEESGIFSQFWAEDFVAVHPEEPTIVVNQSDIDVEVEDLIAEPPDWTEDLQELVDQMGILRSKP

(19) United States
(12) Patent Application Publication
(10) Pub. No.: US 2023/0340520 A1
(43) Pub. Date: Oct. 26, 2023

(54) RESISTANCE GENES AND PLANTS RESISTANT TO BEGOMOVIRUSES

(71) Applicant: RIJK ZWAAN ZAADTEELT EN ZAADHANDEL B.V., De Lier (NL)

(72) Inventors: Florian MÜLLER, De Lier (NL); Adrianus Cornelis KOEKEN, De Lier (NL)

(21) Appl. No.: 18/163,503

(22) Filed: Feb. 2, 2023

Related U.S. Application Data
(63) Continuation-in-part of application No. PCT/EP2021/072412, filed on Aug. 11, 2021.
Application No. PCT/EP2021/072412 is a continuation-in-part of application No. PCT/EP20/72536, filed on Aug. 11, 2020.

(51) Int. Cl. C12N 15/82; C07K 14/415

(52) U.S. Cl. CPC C12N 15/8283; C07K 14/415

(57) ABSTRACT

The present invention relates to a modified YLS9 gene, wherein the modified YLS9 gene can comprise one or more nucleotides replaced, inserted and/or deleted relative to the wild type, and wherein said one or more replaced, inserted and/or deleted nucleotides result in an absence of functional YLS9 protein. According to a further aspect thereof, the invention relates to a modified HsfA2 gene, wherein the modified HsfA2 gene can comprise one or more nucleotides replaced, inserted and/or deleted relative to the wild type, and wherein said one or more replaced, inserted and/or deleted nucleotides result in an absence of functional HsfA2 protein. A plant comprising the modified YLS9 gene and/or the modified HsfA2 gene, preferably both homozygously, the plant exhibits at least intermediate resistance to a Begomovirus, in particular ToLCNDV.

### RELATED APPLICATIONS AND INCORPORATION BY REFERENCE

This application is a continuation-in-part application of international patent application Serial No. PCT/EP2021/072412 filed 11 Aug. 2021, which published as PCT Publication No. WO 2022/034149 on 17 Feb. 2022, which claims benefit of international patent application Serial No. PCT/EP2020/072536 filed 11 Aug. 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been submitted electronically and is hereby incorporated by reference in its entirety. Said XML copy, was created Jan. 27, 2023, is named Y7954-00564SL.xml and is 38,373 bytes in size.

### FIELD OF THE INVENTION

The present invention relates to modified genes conferring resistance against Begomoviruses, in particular ToLCNDV and/or ToLCPMV, and cucumber plants and other plants of the Cucurbitaceae plant family which can comprise those genes. The invention further relates to progeny, seed, and plant parts such as fruits of said resistant plants; and the invention relates to propagation material suitable for producing said plants. The invention also relates to markers for identifying resistant plants, the use of said markers, and to methods for selecting and producing the resistant plants.

### BACKGROUND OF THE INVENTION

Begomoviruses form a genus of viruses in the family Geminiviridae that exhibit a wide host range in a number of economically valuable crop species including those of the Cucurbitaceae (e.g. Cucurbita moschata, Cucurbita pepo, Cucumis melo, Cucumis sativus, Cucurbita maxima, and Citrullus lanatus). Currently there are over 300 species classified as Begomoviruses, including e.g. Tomato Leaf Curl New Delhi Virus (ToLCNDV), Tomato Leaf Curl Palampur Virus (ToLCPMV), Cucurbit Leaf Curl Virus (CuLCV), Melon Chlorotic Leaf Curl Virus (MCLCV), Melon Leaf Curl Virus (MLCV), Squash Leaf Curl Virus (SqLCV) and Cucumber Leaf Crumple Virus (CuLCrV). Begomoviruses are transmitted by an insect vector, which can be the whitefly Bemisia tabaci or other whiteflies. Disease symptoms typically manifest in infected plants as leaf chlorosis, mottled or mosaic leaves, leaf curling or distortion, and stunting of the plant. Fruits grown from Begomovirus infected plants may have symptoms ranging from rough skin, longitudinal cracking, dehydration and speckling. Plants infected with the virus at an early stage may be severely stunted and fruit production may be affected, if not suppressed.

With exceptionally high yield losses attributed to Begomovirus infection, preventing infections from occurring and spreading have become of utmost importance. Currently, control measures against some Begomoviruses are limited and mainly rely on various cultural, phytosanitary and hygienic practices to control whiteflies, including biological control or chemical treatments of whiteflies, cultivation of plants under insect-proof greenhouses, and the elimination of infected plants.

Plant viruses multiply inside their host cells. The genome of Begomoviruses such as ToLCNDV, ToLCPMV, CuLCV, MCLCV, MLCV, SqLCV, and CuLCrV consists of one (monopartite) or two (bipartite) DNA molecules that are individually encapsidated in a virion. Virus and host plant interaction studies have shown that important viral proteins interact with host proteins, leading to the increase of viral DNA. Thus, Begomoviruses heavily rely on the host cell replication machinery for replication and spreading.

Citation or identification of any document in this application is not an admission that such document is available as prior art to the present invention.

### SUMMARY OF THE INVENTION

In the research that led to the invention, cucumber plants (Cucumis sativus L.) and other plants of the Cucurbitaceae plant family resistant to Begomovirusses, in particular ToLCNDV and ToLCPMV, were identified. It was surprisingly found that the resistance resulted from modifications in two different genes, the Yellow Leaf Specific gene 9 (YLS9) and Heat stress transcription factor A2 gene (HsfA2).

The present invention relates to a modified YLS9 gene, the wild type of which has a coding sequence according to SEQ ID NO: 2 encoding a protein having SEQ ID NO: 3 or the wild type of which encodes a protein having at least 70% sequence identity to SEQ ID NO: 3, wherein the modified YLS9 gene can comprise one or more nucleotides replaced, inserted and/or deleted relative to the wild type, and wherein said one or more replaced, inserted and/or deleted nucleotides result in an absence of functional YLS9 protein. According to a further aspect thereof, the invention relates to a modified HsfA2 gene, the wild type of which has a coding sequence according to SEQ ID NO: 7 encoding a protein having SEQ ID NO: 8 or the wild type of which encodes a protein having at least 70% sequence identity to SEQ ID NO: 8, wherein the modified HsfA2 gene can comprise one or more nucleotides replaced, inserted and/or deleted relative to the wild type, and wherein said one or more replaced, inserted and/or deleted nucleotides result in an absence of functional HsfA2 protein. A plant which can comprise the modified YLS9 gene and/or the modified HsfA2 gene, preferably both homozygously, the plant exhibits at least intermediate resistance to a Begomovirus, in particular ToLCNDV.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSITS

Seed of Cucumis sativus L. comprising the modified YLS9 gene of the invention was deposited with NCIMB Ltd, Ferguson Building, Craibstone Estate, Bucksburn, Aberdeen AB21 9YA, UK on 9 Mar. 2020, under deposit accession numbers NCIMB 43586. The seed of NCIMB 43586 comprises the mutations of the modified YLS9 gene as described in Table 3. Seed of cucumber (Cucumis sativus L.) comprising the modified HsfA2 gene of the invention was deposited with NCIMB Ltd, Ferguson Building, Craibstone Estate, Bucksburn, Aberdeen AB21 9YA, UK on 9 Mar. 2020, under deposit accession numbers NCIMB 43587. The seed of NCIMB 43587 comprises the mutations of the modified HsfA2 gene as described in Table 3.

The Deposits with NCIMB Ltd., under deposit accession number ACCESSION NOS. 43586 and 43587 were made and accepted pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1A-1E—Examples of leaves of cucumber plants in a young plant test classified according to the scale as presented in Table 2. All pictures show all leaves of one individual in the young plant test as described in Example 1. This not only illustrates symptoms on the leaves, but also the reduction in size and number of leaves. 1a) leaves of plant scored 1 (completely resistant); b) leaves of plant scored 3 (intermediate resistant); c) leaves of plant scored 5 (susceptible); d) leaves of plant scored 7 (susceptible); e) leaves of plant scored 9 (susceptible).

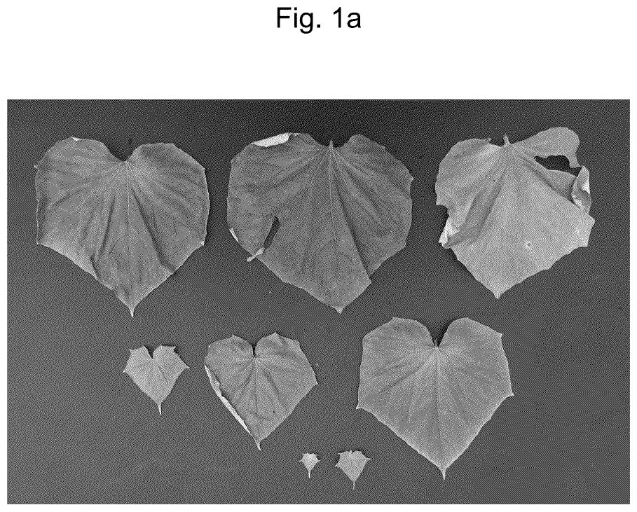

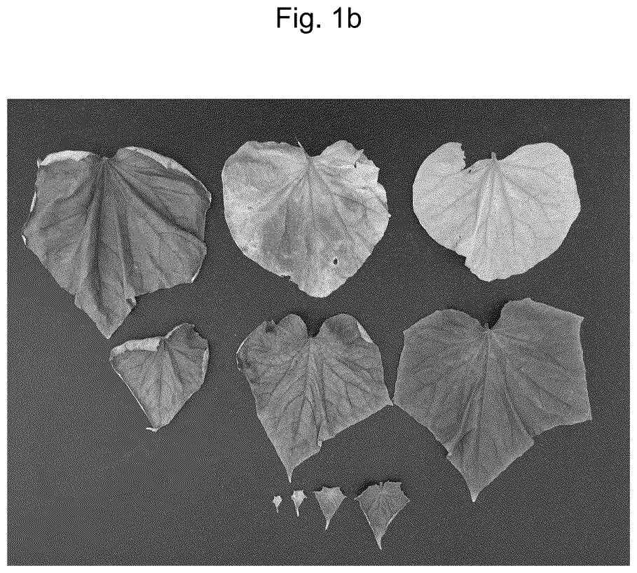

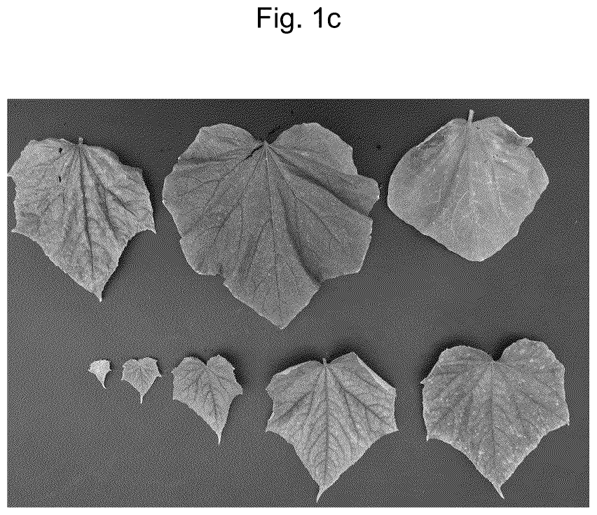

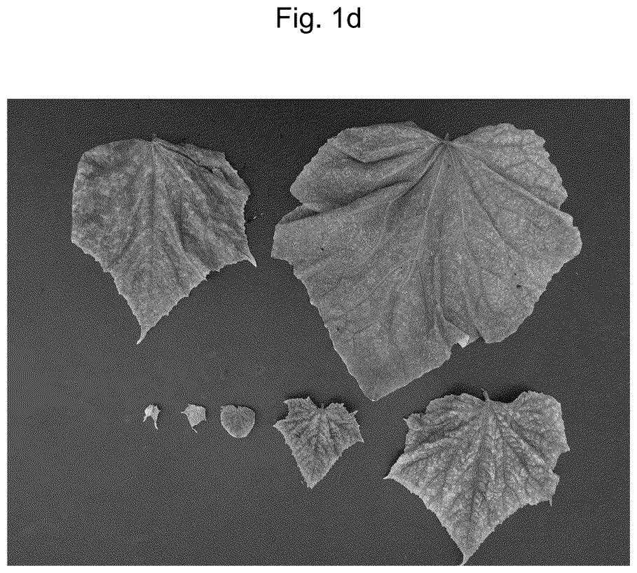

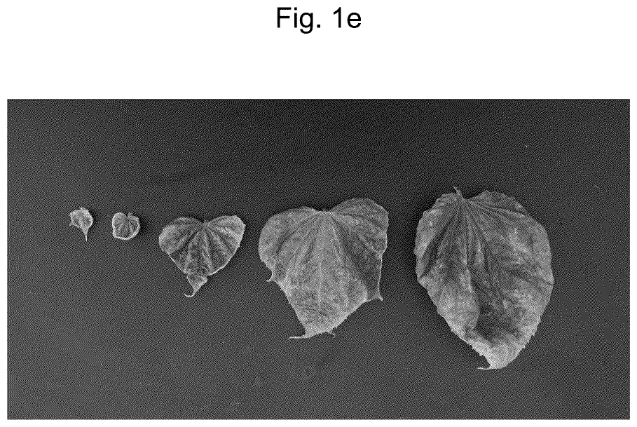

### DETAILED DESCRIPTION OF THE INVENTION

The wild type YLS9 gene encodes a protein whose sequence is similar to tobacco hairpin-induced gene (HIN1) and Arabidopsis non-race specific disease resistance gene (NDR1). Expression of this gene in Arabidopsis thaliana is induced by e.g. Cucumber Mosaic Virus, spermine and during senescence. The protein can comprise a Late Embryogenesis Abundant (LEA) domain. LEA proteins have been found to accumulate to high levels during the last stage of seed formation (when a natural desiccation of the seed tissues takes place) and during periods of water deficit in vegetative organs.

In the publicly available genome assembly of Cucumis sativus L. var. sativus cv. 9930 version 3 (Qing Li et al. (2019) A chromosome-scale genome assembly of cucumber (Cucumis sativus L.), GigaScience Vol 8(6) giz072), the wild type YLS9 gene in Cucumis sativus is located on chromosome 1 at position 9631802 . . . 9632999.

The present invention provides a modified YLS9 gene, the wild type of which has a coding sequence according to SEQ ID NO: 2 encoding a protein having SEQ ID NO: 3 or the wild type of which encodes a protein having in order of increased preference at least 70%, 75%, 80%, 85%, 90%, 91% 92%, 93%, 94%, 95%, 96%, 97%, 98%, 99% sequence identity to SEQ ID NO: 3, wherein the modified YLS9 gene can comprise one or more nucleotides replaced, inserted and/or deleted relative to the wild type, and wherein said one or more replaced, inserted and/or deleted nucleotides result in an absence of functional YLS9 protein. The wild type coding sequence according to SEQ ID NO: 2 is the sequence encoding the YLS9 protein of Cucumis sativus. The wild type amino acid sequence according to SEQ ID NO: 3 is the sequence of the YLS9 protein of Cucumis sativus.

The present invention further provides a modified YLS9 gene, the wild type of which has a coding sequence which can comprise SEQ ID NO: 11 encoding a protein which can comprise SEQ ID NO: 12 or the wild type of which encodes a protein having in order of increased preference at least 70%, 75%, 80%, 85%, 90%, 91% 92%, 93%, 94%, 95%, 96%, 97%, 98%, 99% sequence identity to SEQ ID NO: 12, wherein the modified YLS9 gene can comprise one or more nucleotides replaced, inserted and/or deleted relative to the wild type, and wherein said one or more replaced, inserted and/or deleted nucleotides result in an absence of functional YLS9 protein. The wild type coding sequence according to SEQ ID NO: 11 is the sequence encoding the YLS9 protein of Cucumis melo. The wildtype amino acid sequence according to SEQ ID NO: 12 is the sequence of the YLS9 protein of Cucumis melo.

The YLS9 gene of the invention thus has the same sequence as a gene that encodes the wildtype YLS9 protein except for the modification. When the gene of the invention encodes a wildtype protein having at least 70% sequence identity with SEQ ID NO:3 or SEQ ID NO:12, the modification is not included in the differences between the wildtype SEQ ID NO:3 or SEQ ID NO:12 that lead to the percentage identity.

In the context of this invention, the wild type YLS9 gene also encompasses a gene which has a genomic sequence and coding sequence, that in order of increased preference, has 70%, 75%, 80%, 85%, 90%, 91% 92%, 93%, 94%, 95%, 96%, 97%, 98%, 99%, 100% sequence identity to SEQ ID NO: 1 and SEQ ID NO: 2, respectively.

The wild type YLS9 gene of the invention further encompasses a gene encoding a YLS9 protein which can comprise SEQ ID NO: 3 or a protein that has, in order of increased preference 70%, 75%, 80%, 85%, 90%, 91% 92%, 93%, 94%, 95%, 96%, 97%, 98%, 99% sequence identity to SEQ ID NO: 3.

As used herein, sequence identity is the percentage of nucleotides or amino acids that is identical between two sequences after proper alignment of those sequences. The person skilled in the art is aware of how to align sequences, for example by using a sequence alignment tool such as BLAST®, which can be used for both nucleotide sequences and protein sequences. To obtain the most significant result, the best possible alignment that gives the highest sequence identity score should be obtained. The percentage sequence identity is calculated through comparison over the length of the shortest sequence in the assessment, whereby in the present case a sequence represents a gene that at least can comprise a start codon and a stop codon, or a complete protein encoded by such a gene.

The modified YLS9 gene of the invention can comprise one or more nucleotides replaced, inserted and/or deleted relative to the wild type, and said one or more replaced, inserted and/or deleted nucleotides result in an absence of functional YLS9 protein. The absence of functional protein can have but is not limited to one of the following causes. The absence of functional YLS9 protein can be due to the absence of YLS9 RNA or a significantly decreased YLS9 RNA level, resulting in a complete absence or a reduced and biologically inadequate level of YLS9 protein. The absence of functional YLS9 protein can also mean an absence or non-functionality of one or more of the functional domains of the YLS9 protein, resulting in a modified YLS9 protein that cannot perform its function. The absence of functional YLS9 protein can further mean that the modified protein has gained certain amino acids, destroying the wild type functionality of the protein. More specifically, the absence of functional protein can further mean that the protein has lost a protein-protein and/or protein-DNA interaction site.

Therefore, the present invention provides a modified YLS9 gene wherein the modified gene can comprise a mutation in SEQ ID NO: 2, or in a homologous sequence having in order of increased preference at least 70%, 75%, 80%, 85%, 90%, 91% 92%, 93%, 94%, 95%, 96%, 97%, 98%, 99% sequence identity to SEQ ID NO: 2, wherein said mutation causes the loss of a protein-protein and/or protein-DNA interaction site in SEQ ID NO: 3 or in a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 3.

The present invention provides a modified YLS9 gene wherein the modified gene can comprise a frameshift mutation, in particular a frameshift mutation caused by a deletion of an adenine on position 551 in SEQ ID NO: 2, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO: 2.

The present invention also provides a modified YLS9 gene, wherein the modified gene can comprise a nucleotide substitution on position 76 of SEQ ID NO: 2, wherein a cytosine is replaced by an adenine, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO: 2, or wherein the modified gene encodes a protein having an amino acid replacement on position 26 of SEQ ID NO: 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 3.

In particular, the present invention also provides a modified YLS9 gene, wherein the modified gene encodes a protein having an amino acid replacement of Glutamine to Lysine on position 26 of SEQ ID NO: 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 3.

The invention further provides a modified YLS9 gene which can comprise both a frameshift mutation caused by a deletion of an adenine on position 551 in SEQ ID NO: 2, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO: 2, and further which can comprise a nucleotide substitution on position 76 of SEQ ID NO: 2, wherein the cytosine is preferably replaced by an adenine, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO: 2, or wherein the modified gene encodes a protein having an amino acid replacement, on position 26 of SEQ ID NO: 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 3.

In particular, the invention provides a modified YLS9 gene which can comprise both a frameshift mutation caused by a deletion of an adenine on position 551 in SEQ ID NO: 2, or on a corresponding position of a homologous sequence having in order of increased preference at least 70%, 75%, 80%, 85%, 90%, 91% 92%, 93%, 94%, 95%, 96%, 97%, 98%, 99% sequence identity to SEQ ID NO: 2, and wherein the modified gene encodes a protein which can comprise an amino acid replacement of Glutamine to Lysine on position 26 of SEQ ID NO: 3 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 3.

In one embodiment, the modified YLS9 gene of the invention can comprise a coding sequence having SEQ ID NO: 4, or a sequence encoding a protein having SEQ ID NO: 5.

The invention further relates to a modified YLS9 gene which can comprise a mutation leading to a premature stop codon, wherein the premature stop codon leads to absence of functional YLS9 protein. Preferably, the premature stop codon is located within or before the part encoding the LEA2 domain of the YLS9 protein.

Therefore, the present invention also provides a modified YLS9 gene, wherein the modified gene can comprise, in order of increased preference, a premature stop codon on or before position 505, 480, 455, 430, 405, 380, 355, 330, 305, 280, 273 of SEQ ID NO: 2, or wherein the modified YLS9 protein is truncated, in order of increased preference, on or before position 175, 165, 155, 145, 135, 125, 115, 105, or 93 of SEQ ID NO: 3.

The modified YLS9 gene of the invention, when homozygously present in a plant, in particular a plant of the Cucurbitaceae plant family and more in particular a cucumber plant, confers resistance against a Begomovirus, in particular against ToLCNDV and/or ToLCPMV.

The wild type of the second gene that is identified to provide Begomovirus resistance, the HsfA2 gene, encodes a heat shock transcription factor. Heat Shock transcription Factors (HSFs) are mainly involved in the activation of genes in response to heat stress as well as other abiotic and biotic stresses. Compared to animals, in plants HSF gene families which can comprise many individual HSF members have been identified. This is probably due to the fact that plants are sessile organisms and cannot escape to different grounds when confronted with unfavorable stresses, forcing plants to develop a complex network of stress response mechanisms during the course of evolution. Based on their structural properties HSFs in plants have been classified into three different classes: HSFA, HSFB and HSFC.

In the publicly available genome assembly of Cucumis sativus L. var. sativus cv. 9930 version 3 (Qing Li et al. (2019) A chromosome-scale genome assembly of cucumber (Cucumis sativus L.), GigaScience Vol. 8(6) giz072), the wild type HsfA2 gene in Cucumis sativus is located on chromosome 2 at position 17542329 . . . 17543717.

The present invention provides a modified HsfA2 gene the wild type of which has a coding sequence according to SEQ ID NO: 7 encoding a protein having SEQ ID NO: 8 or the wild type of which encodes a protein having in order of increased preference at least 70%, 75%, 80%, 85%, 90%, 91% 92%, 93%, 94%, 95%, 96%, 97%, 98%, 99% sequence identity to SEQ ID NO: 8, wherein the modified HsfA2 gene can comprise one or more nucleotides replaced, inserted and/or deleted relative to the wild type, and wherein said one or more replaced, inserted and/or deleted nucleotides result in an absence of functional HsfA2 protein. The wild type coding sequence according to SEQ ID NO: 7 is the sequence encoding the HsfA2 protein of Cucumis sativus. The wildtype amino acid sequence according to SEQ ID NO: 8 is the sequence of the HsfA2 protein of Cucumis sativus.

The present invention further provides a modified HsfA2 gene the wild type of which has a coding sequence which can comprise SEQ ID NO: 13 encoding a protein which can comprise SEQ ID NO: 14 or the wild type of which encodes a protein having in order of increased preference at least 70%, 75%, 80%, 85%, 90%, 91% 92%, 93%, 94%, 95%, 96%, 97%, 98%, 99% sequence identity to SEQ ID NO: 14, wherein the modified HsfA2 gene can comprise one or more nucleotides replaced, inserted and/or deleted relative to the wild type, and wherein said one or more replaced, inserted and/or deleted nucleotides result in an absence of functional HsfA2 protein. The wild type coding sequence according to SEQ ID NO: 13 is the sequence encoding the HsfA2 protein of Cucumis melo. The wildtype amino acid sequence according to SEQ ID NO: 14 is the sequence of the HsfA2 protein of Cucumis melo.

The HsfA2 gene of the invention thus has the same sequence as a gene that encodes the wildtype HsfA2 protein except for the modification. When the gene of the invention encodes a wildtype protein having at least 70% sequence identity with SEQ ID NO:8 or SEQ ID NO: 14, the modification is not included in the differences between the wildtype SEQ ID NO: 8 or SEQ ID NO: 14 that lead to the percentage identity.

The wild type HsfA2 gene of the invention also encompasses a gene which has a genomic sequence and coding sequence, that in order of increased preference, has 70%, 75%, 80%, 85%, 90%, 91% 92%, 93%, 94%, 95%, 96%, 97%, 98%, 99%, 100% sequence identity to SEQ ID NO: 6 and 7, respectively.

The wild type HsfA2 gene of the invention further encompasses a gene encoding a protein which can comprise SEQ ID NO: 8 or a protein that has, in order of increased preference 70%, 75%, 80%, 85%, 90%, 91% 92%, 93%, 94%, 95%, 96%, 97%, 98%, 99% sequence identity to SEQ ID NO: 8.

The modified HsfA2 gene of the invention can comprise one or more nucleotides replaced, inserted and/or deleted relative to the wild type, and said one or more replaced, inserted and/or deleted nucleotides result in an absence of functional HsfA2 protein. The absence of functional protein can have, but is not limited to, one of the following causes. The absence of functional HsfA2 protein can be due to the absence of HsfA2 RNA or a significantly decreased HsfA2 RNA level, resulting in a complete absence or a reduced and biologically inadequate level of HsfA2 protein. The absence of functional HsfA2 protein can also mean an absence of one or more of the functional domains of the HsfA2 protein, resulting in a modified HsfA2 protein that cannot perform its function, or is not recognized by the pathogen anymore.

The present invention provides a modified HsfA2 gene which can comprise one of the following mutations or any combination thereof:
a) a nucleotide substitution on position 1084 in SEQ ID NO: 7 wherein a thymine is replaced by a cytosine, or on a corresponding position of a homologous nucleotide sequence having in order of increased preference at least 70%, 75%, 80%, 85%, 90%, 91% 92%, 93%, 94%, 95%, 96%, 97%, 98%, 99% sequence identity to SEQ ID NO: 7, or wherein the modified gene encodes a protein which can comprise an amino acid replacement on position 362 of SEQ ID NO: 8 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8;
b) a deletion of a triplet encoding a Glutamic acid on position 265 in SEQ ID NO: 8, or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8;
c) a nucleotide substitution on position 561 in SEQ ID NO: 7 where a thymine is replaced by a guanine, or on a corresponding position of a homologous nucleotide sequence having in order of increased preference at least 70%, 75%, 80%, 85%, 90%, 91% 92%, 93%, 94%, 95%, 96%, 97%, 98%, 99% sequence identity to SEQ ID NO: 7, or wherein the modified gene encodes a protein which can comprise an amino acid replacement on position 187 of SEQ ID NO: 8 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8;
d) a deletion of a triplet encoding a Serine on position 22 in SEQ ID NO: 8, or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8.

In particular, the amino acid replacement on position 362 of SEQ ID NO: 8 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8, is a replacement of a Serine by a Proline.

In particular, the amino acid replacement on position 187 of SEQ ID NO: 8 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8, is a replacement of an Aspartic acid by a Glutamic acid.

The amino acid substitution of a Serine by a Proline on position 362 of SEQ ID NO: 8 severely reduces side chain flexibility, and while Serine could interact with other biomolecules with potentially three hydrogen bonds and other van der Waals bonds, Proline can only interact with van der Waals bonds. Therefore, this mutation seems to have a severe impact on the wildtype functionality of the HsfA2 gene.

In a preferred embodiment, the modified HsfA2 gene can comprise at least a nucleotide substitution on position 1084 in SEQ ID NO: 7 wherein a thymine is replaced by a cytosine, or on a corresponding position of a homologous nucleotide sequence having in order of increased preference at least 70%, 75%, 80%, 85%, 90%, 91% 92%, 93%, 94%, 95%, 96%, 97%, 98%, 99% sequence identity to SEQ ID NO: 7, or wherein the modified gene encodes a protein which can comprise an amino acid replacement of Serine to Proline on position 362 of SEQ ID NO: 8 or on a corresponding position of a homologous amino acid sequence having in order of increased preference at least 70%, 75%, 80%, 85%, 90%, 91% 92%, 93%, 94%, 95%, 96%, 97%, 98%, 99% sequence identity to SEQ ID NO: 8.

In a one embodiment, the modified HsfA2 gene can comprise a coding sequence having SEQ ID NO: 9, or a sequence encoding a protein having SEQ ID NO: 10.

The modified HsfA2 gene when homozygously present in a plant, in particular a plant of the Cucurbitaceae plant family and more in particular a cucumber plant, confers resistance against a Begomovirus, in particular against ToLCNDV and/or ToLCPMV.

In one embodiment, the modified YLS9 gene and/or HsfA2 gene of this invention is a nucleic acid, in particular a nucleic acid molecule, more in particular an isolated nucleic acid molecule.

The invention further relates to a plant, preferably a plant of the Cucurbitaceae plant family, more preferably a Cucumis sativus plant or a Cucumis melo plant, most preferably a Cucumis sativus plant, wherein the plant can comprise the modified YLS9 gene as described in the present application in its genome, or wherein the plant can comprise the modified HsfA2 gene as described in the present application in its genome, or wherein the plant can comprise both modified genes of the invention in its genome. All these plants are referred to herein as a ‘plant of the invention’.

In a further embodiment, the plant of the invention is an agronomically elite plant, preferably an agronomically elite Cucumis melo plant or a Cucumis sativus plant, most preferably a Cucumis sativus plant.

In the context of this invention, an agronomically elite plant is a plant having a genotype that, as a result of human intervention, can comprise an accumulation of distinguishable and desirable agronomic traits which allow a producer to harvest a product of commercial significance, preferably the agronomically elite plant of the invention is a plant of an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant, which is the result of a cross between two different plants having different genotypes. More in particular, a hybrid plant is the result of a cross between plants of two different inbred lines, such a hybrid plant may e.g. be a plant of an F₁ hybrid variety. A hybrid plant of the invention is preferably the result of a cross between two different plants of the same species having different genotypes but which both have the modified gene or genes of the invention.

In one embodiment, the agronomically elite Cucumis sativus plant of the invention is gynoecious plant, or a monoecious plant having in order of increased preference at least 50%, 60%, 70%, 80%, 90%, 95% gynoecious flowers.

In one embodiment the agronomically elite melon plant of the invention belongs to the subspecies Cucumis melo subsp. melo.

Each of the two modified genes of the invention on its own, provided that it is homozygously present in the genome of a plant, provides at least intermediate resistance to a Begomovirus, in particular ToLCNDV.

Each of the two modified genes of the invention on its own, provided that it is homozygously present in the genome of a plant, provides complete resistance to the Begomovirus, ToLCPMV.

Therefore, the invention relates to a plant, which only has the modified YLS9 gene of the invention homozygously present in its genome, providing at least intermediate resistance against a Begomovirus in particular ToLCNDV.

The invention further relates to a plant, which only has the modified YLS9 gene of the invention homozygously present in its genome, providing complete resistance against a ToLCPMV.

The invention thus also relates to a plant which only has the modified HsfA2 gene of the invention homozygously present in its genome, providing at least intermediate resistance against a Begomovirus, in particular ToLCNDV.

The invention thus also relates to a plant, which only has the modified HsfA2 gene of the invention homozygously present in its genome, providing complete resistance against a Begomovirus, in particular ToLCPMV.

In a further embodiment, the plant of the invention carrying only the modified YLS9 gene of the invention homozygously exhibits at least intermediate resistant against ToLCNDV and complete resistance to Tomato Leaf Curl Palampur Virus (ToLCPMV).

In yet a further embodiment, the plant of the invention carrying only the modified YLS9 gene of the invention homozygously exhibits at least intermediate resistance against ToLCNDV, ToLCPMV, and SqLCV.

In one embodiment, the plant of the invention, carrying only the modified HsfA2 gene of the invention homozygously, exhibits at least intermediate resistance against ToLCNDV and complete resistance to ToLCPMV.

In yet a further embodiment, the plant of the invention, carrying only the modified HsfA2 gene of the invention homozygously, exhibits at least intermediate resistance against ToLCNDV, ToLCPMV, and SqLCV.

The plant of the invention can also comprise both the modified YLS9 gene and the modified HsfA2 gene homozygously. When both modified genes of the invention are homozygously present they provide complete resistance against a Begomovirus, in particular ToLCNDV.

In a further embodiment, the plant of the invention, carrying both modified genes of the invention homozygously, exhibits complete resistant against ToLCNDV and ToLCPMV.

In yet a further embodiment, the plant of the invention, carrying both modified genes of the invention homozygously, exhibits complete resistance against ToLCNDV, ToLCPMV, and SqLCV.

Seed of Cucumis sativus L. which can comprise the modified YLS9 gene of the invention and the wild type HsfA2 gene homozygously were deposited with the NCIMB under accession number NCIMB 43586.

Seed of Cucumis sativus L. which can comprise the modified HsfA2 gene of the invention and the wild type YLS9 gene homozygously were deposited with the NCIMB under accession number NCIMB 43587.

The invention thus relates to plants grown from seed deposited under NCIMB accession numbers NCIMB 43586. The invention also relates to plants grown from seed deposited under NCIMB accession numbers NCIMB 43587.

As used herein, resistance or susceptibility against ToLCNDV is determined in a young plant test. Young plants of each of the genotypes are mechanically inoculated with ToLCNDV. Mechanical inoculation of ToLCNDV is performed using the method adapted from Lopez et al. 2015, such that the ToLCNDV inoculum was prepared using buffer (i) as described (Euphytica. 2015 (204): 679-691). The ToLCNDV disease test is performed in a greenhouse with a daytime/night time temperature regime of 20° C./18° C. Young plants are mechanically inoculated twice, at 7 and 9 days after sowing. A final assessment is done 24 days post sowing, by visual scoring for the number of ToLCNDV symptoms, based on the scale described in Table 2. The same disease test can be used to assess resistance or susceptibility against ToLCPMV. Symptoms of ToLCPMV are also scored based on the scale described in Table 2. A suitable negative control in the described disease test should be a plant scoring 5 or higher on the scale described in Table 2.

As used herein, a plant exhibiting complete resistance is a plant that, when exposed to the above described disease test, shows no symptoms at all, or a plant showing some non-specific yellowing due to aging, maturation or yellowing not related to viral infection (See Table 2).

As used herein, a plant exhibiting intermediate resistance is a plant that, when exposed to the above described disease test, shows no leaf deformation, symptoms starting to develop mainly on older leaves, some yellowing spots may occur on less than 25% of the plant surface, and re-growth and the top of the plant is symptomless; or a plant showing no leaf deformation, yellowing symptoms affecting 25-50% of the plant, yellow spots are more abundant than score 3, and re-growth and the top of the plant is symptomless (See Table 2).

In the context of this invention the term ‘resistance’ on its own includes both ‘complete resistance’ and ‘intermediate resistance’ to a Begomovirus, in particular to ToLCNDV and/or ToLCPMV.

In a particular embodiment, the resistance conferred by the modified YLS9 gene and/or the modified HsfA2 gene of the invention is against an isolate of ToLCNDV gathered in the Mediterranean or the Middle East.

In another particular embodiment, the resistance conferred by the modified YLS9 gene and/or the modified HsfA2 gene of the invention is against an isolate of ToLPMV gathered in the Middle East. A plant which can comprise only the modified YLS9 gene of the invention or only the modified HsfA2 gene of the invention homozygously will in the above described disease test score 4, 3, 2, or 1 when tested for ToLCNDV. A plant which can comprise both the modified YLS9 gene and the modified HsfA2 gene of the invention homozygously will in the above described disease test score 1 or 2 when tested for ToLCNDV. A plant of the invention will exhibit resistance to ToLCNDV already in the young plant stage.

A plant which can comprise only the modified YLS9 gene of the invention or only the modified HsfA2 gene of the invention homozygously will in the above described disease test already score 2 or 1 when tested for ToLCPMV. A plant of the invention will exhibit resistance to ToLCPMV, already in the young plant stage.

Another aspect of the invention relates to a seed capable of growing into a plant of the invention wherein said plant can comprise the modified YLS9 gene and/or modified HsfA2 gene of the invention, preferably in homozygous state. The invention also relates to use of said seed for the production of a plant of the invention, by growing said seed into a plant.

Yet another aspect of the invention relates to a fruit harvested from a plant of the invention wherein said fruit can comprise the modified YLS9 gene and/or modified HsfA2 gene of the invention, preferably in homozygous state.

The invention also relates to propagation material suitable for producing a plant of the invention, wherein the propagation material is suitable for sexual reproduction, and is in particular selected from a microspore, a pollen, an ovary, an ovule, an embryo sac and an egg cell, or is suitable for vegetative reproduction, and is in particular selected from a cutting, a root, a stem a cell, and a protoplast, or is suitable for tissue culture of regenerable cells or protoplasts, and is in particular selected from a leaf, a pollen, an embryo, a cotyledon, a hypocotyl, a meristematic cell, a root, a root tip, an anther, a flower, a seed and a stem, wherein the propagation material can comprise the modified YLS9 gene and/or the modified HsfA2 gene of the invention.

The invention further relates to a cell of a plant of the invention. Such a cell may either be in isolated form or a part of the complete plant or parts thereof and still forms a cell of the invention because such a cell can comprise the modified YLS9 gene and/or the modified HsfA2 gene of the invention in its genome. Each cell of a plant of the invention carries the modified YLS9 gene and/or the modified HsfA2 gene of the invention. A cell of the invention may also be a regenerable cell that can regenerate into a new plant of the invention.

The invention further relates to plant tissue of a plant of the invention, which can comprise the modified YLS9 gene and/or the modified HsfA2 gene of the invention. The tissue can be undifferentiated tissue or already differentiated tissue. Undifferentiated tissue is for example a stem tip, an anther, a petal, or pollen, and can be used in micropropagation to obtain new plantlets that are grown into new plants of the invention. The tissue can also be grown from a cell of the invention.

The invention further relates to a method for the production of a plant which can comprise the modified YLS9 gene and/or the modified HsfA2 gene of the invention, which plant is resistant to a Begomovirus, in particular to ToLCNDV and/or ToLCPMV, by using tissue culture or by using vegetative propagation.

The invention moreover relates to progeny of a plant, a cell, a tissue, or a seed of the invention, which progeny can comprise the modified YLS9 gene and/or the modified HsfA2 gene of the invention. Such progeny can in itself be a plant, a cell, a tissue, or a seed. The progeny can in particular be progeny of a plant of the invention deposited under NCIMB number 43586 or NCIMB 43587. As used herein, progeny can comprise the first and all further descendants from a cross with a plant of the invention, wherein a cross can comprise a cross with itself or a cross with another plant, and wherein a descendant that is determined to be progeny can comprise the modified YLS9 gene and/or the modified HsfA2 gene of the invention. Descendants can be obtained through selfing and/or further crossing of the deposit. Progeny also encompasses material that is obtained by vegetative propagation or another form of multiplication.

The invention further relates to the germplasm of plants of the invention. The germplasm is constituted by all inherited characteristics of an organism and according to the invention encompasses at least the trait of the invention. The germplasm can be used in a breeding program for the development of plants that exhibits resistance to a Begomovirus, in particular ToLCNDV and/or ToLCPMV. The use of germplasm that can comprise the modified YLS9 gene and/or the modified HsfA2 gene of the invention in breeding is also part of the present invention.

The invention also relates to the use of the modified YLS9 gene and/or the modified HsfA2 gene of the invention for producing a plant that is resistant to a Begomovirus, in particular ToLCNDV and/or ToLCPMV. The plant is preferably a plant that belongs to the Cucurbitaceae plant family, in particular a Cucumis sativus L. plant.

The current invention also relates to the use of a plant of the invention as a crop, as a source of seed or as a source of propagation material.

The invention also relates to a marker for the identification of the modified YLS9 gene of the invention which marker can comprise any of the modifications in the modified YLS9 gene as described herein and can thereby identify said modifications. In particular, a marker for the identification of the modified YLS9 gene of the invention detects a substitution from a cytosine to a adenine on position 76 of the wild type YLS9 gene sequence of SEQ ID NO: 2, or the marker detects a deletion of an adenine on position 551 of the wild type YLS9 gene sequence of SEQ ID NO: 2, or the marker detects any of the above described modifications on a corresponding position of a homologous sequence that in order of increased preference, has 70%, 75%, 80%, 85%, 90%, 91% 92%, 93%, 94%, 95%, 96%, 97%, 98%, 99% sequence identity to SEQ ID NO: 2. Suitably, the marker can comprise a substitution from a cytosine to a adenine on position 76 of the wild type YLS9 gene sequence of SEQ ID NO: 2, or a deletion of an adenine on position 551 of the wild type YLS9 gene sequence of SEQ ID NO: 2 or in a homologous sequence that has at least 70% sequence identity with SEQ ID NO: 2.

The invention further relates to a marker for the identification of the modified HsfA2 gene of the invention which marker can comprise any of the modifications in the modified HsfA2 gene as described herein and can thereby identify said modifications. In particular, a marker for the identification of the modified HsfA2 of the invention detects a deletion of a triplet CTT on position 65-67 of the wild type HsfA2 gene sequence of SEQ ID NO: 7, or the marker detects a deletion of a triplet AGA on position 792-794 of the wild type HsfA2 gene sequence of SEQ ID NO: 7, or the marker detects a substitution from a thymine to a guanine on position 561 of the wild type HsfA2 gene sequence of SEQ ID NO: 7, or the marker detects a substitution from a thymine to a cytosine on position 1084 of the wild type HsfA2 gene sequence of SEQ ID NO: 7, or the marker detects any of the above described modifications on a corresponding position of a homologous sequence that in order of increased preference, has 70%, 75%, 80%, 85%, 90%, 91% 92%, 93%, 94%, 95%, 96%, 97%, 98%, 99% sequence identity to SEQ ID NO: 7. Suitably, the marker can comprise a deletion corresponding to a deletion of a triplet CTT on position 65-67 of the wild type HsfA2 gene sequence of SEQ ID NO: 7, or corresponding to a deletion of a triplet AGA on position 792-794 of the wild type HsfA2 gene sequence of SEQ ID NO: 7, or can comprise a substitution corresponding to a substitution from a thymine to a guanine on position 561 of the wild type HsfA2 gene sequence of SEQ ID NO: 7, or a substitution corresponding to a substitution from a thymine to a cytosine on position 1084 of the wild type HsfA2 gene sequence of SEQ ID NO: 7 or in a homologous sequence that has at least 70% sequence identity with SEQ ID NO: 7.

The use of a marker described herein for identification of the modified YLS9 or the modified HsfA2 of the invention is also part of this invention.

The present invention relates to a method for identification of a plant which can comprise the modified YLS9 gene and/or HsfA2 gene of the invention, which plant shows resistance to a Begomovirus, in particular to ToLCNDV and/or ToLCPMV, wherein the identification can comprise determining the presence of a modification in the YLS9 gene and/or HsfA2 gene, or in a homologous sequence thereof, and analyzing if the plant which can comprise the modification exhibits resistance to a Begomovirus, in particular to ToLCNDV and/or ToLCPMV.

Determining the presence of a modification in the modified YLS9 gene and/or HsfA2 gene of the invention can comprise identification of any modification in SEQ ID NO: 1 or SEQ ID NO: 6 that leads to Begomovirus resistance, in particular to ToLCNDV resistance and/or ToLCPMV resistance. Determining the presence of a modification includes determining the presence of any of the modifications as described herein, in particular those presented in Table 3. Determining the presence of a modification can be done through sequence comparison, which is known to the skilled person. Determining a modification is suitably done by using a marker that is designed to identify such modification as its sequence can comprise that specific modification, in particular using a marker as described herein. Alternatively, determining the presence of a modification in the modified YLS9 gene and/or HsfA2 gene of the invention is done on the protein level and can comprise identification of any modification in SEQ ID NO: 3 or SEQ ID NO: 8. This is suitably done using Western Blotting.

The invention further relates to a method for selecting a plant that shows resistance to a Begomovirus, in particular TOLCNDV and/or ToLCPMV, which can comprise identifying the presence of a modification in the YLS9 gene and/or HsfA2 gene of the invention, and selecting a plant which can comprise a modification in one or both genes as a Begomovirus resistant plant, in particular a ToLCNDV and/or ToLCPMV resistant plant. Optionally, the method can comprise a further step in which virus resistance is determined, for example by performing the disease test as described in Example 1. The selected plant obtained by the selection method is also a part of this invention.

The invention further relates to a method for seed production which can comprise growing a plant from a seed of the invention that can comprise the modified YLS9 gene and/or modified HsfA2 gene of the invention preferably homozygously, allowing the plant to produce a fruit with seed, harvesting the fruit, and extracting those seed. Production of the seed is suitably done by selfing or by crossing with another plant that is optionally also a plant of the invention. Preferably, the plant grown from the seed produced as described herein is resistant to a Begomovirus, in particular to ToLCNDV and/or ToLCPMV.

The invention also relates to a method for producing hybrid seed, which can comprise crossing a first parent plant with a second parent plant and harvesting the resultant hybrid seed, wherein the first parent plant and/or the second parent plant is a plant of the invention which can comprise the modified YLS9 gene and/or modified HsfA2 gene of the invention. Preferably, both parent plants are homozygous for the modified YLS9 gene or modified HsfA2 gene of the invention. It is even more preferred that both parent plants are homozygous for both modified genes of the invention.

The invention also relates to the hybrid seed produced by the method described herein and a hybrid plant grown from said hybrid seed. Preferably, the hybrid plant grown out of the hybrid seed can comprise one or both of the two modified genes of the invention homozygously.

The present invention relates to a method for producing a plant that is resistant to a Begomovirus, in particular to ToLCNDV and/or ToLCPMV, which can comprise introducing a modification in an YLS9 gene and/or HsfA2 gene, which modification leads to resistance. Said method can comprise the introduction of a deletion, a substitution, or an insertion in the coding sequence of an YLS9 gene and/or HsfA2 gene. The introduction of such a modification can be done by a random mutagenesis approach using a chemical compound, such as ethyl methane sulphonate (EMS); or by using physical means, such as UV-irradiation, fast neutron exposure, or other irradiation techniques.

A modification in the YLS9 gene and/or the HsfA2 gene can also be introduced via more specific, so-called site-directed approach, such as targeted methods like homologous recombination, oligonucleotide-based mutation introduction, zinc-finger nucleases (ZFN), transcription activator-like effector nucleases (TALENs) or Clustered Regularly Interspaced Short Palindromic Repeat (CRISPR) systems.

In one embodiment, the plant of the invention can comprise the modified YLS9 gene and/or modified HsfA2 gene of the invention, preferably in homozygous state, wherein the modification in the YLS9 gene and/or HsfA2 gene is non-naturally occurring.

In a further embodiment, the plant of the invention can comprise the modified YLS9 gene and/or modified HsfA2 gene of the invention, preferably in homozygous state, wherein the modification of the YLS9 gene and/or HsfA2 gene is the result of humanly induced mutagenesis, wherein the induced mutagenesis can be a form of random mutagenesis, or a form of site-directed mutagenesis, in particular a TALENs or CRISPR system.

Transgenic techniques used for transferring sequences between plants that are sexually incompatible can also be used to produce a plant of the invention, by transferring the modified YLS9 gene and/or HsfA2 gene of the invention from one species to another. Techniques that can suitably be used comprise general plant transformation techniques known to the skilled person, such as the use of an Agrobacterium-mediated transformation method. A plant of the deposits or a descendant thereof is a suitable source of the modified genes.

Introduction of the modified YLS9 gene and/or modified HsfA2 gene of the invention can also be done through introgression from a plant which can comprise said modified YLS9 gene and/or modified HsfA2 gene, for example from a plant that was deposited as NCIMB 43586 and/or NCIMB 43587, or from progeny thereof, or from another plant that is resistant to a Begomovirus, in particular to ToLCNDV and/or ToLCPMV, and in which the modified YLS9 gene and/or modified HsfA2 gene was identified. Breeding methods such as crossing and selection, backcrossing, recombinant selection, or other breeding methods that result in the transfer of a genetic sequence from a resistant plant to a susceptible plant can be used. A resistant plant can be of the same species or of a different and/or wild species. Difficulties in crossing between species can be overcome through techniques known in the art such as embryo rescue, or cis-genesis can be applied. Progeny of a deposit can be sexual or vegetative descendants of that deposit, which can be selfed and/or crossed, and can be of an F1, F2, or further generation as long as the descendants of the deposit still comprise the modified gene the invention as present in seed of that deposit. A plant produced by such method is also a part of the invention.

The invention also relates to a method for the production of a plant exhibiting resistance against a Begomovirus, in particular ToLCNDV and/or ToLCPMV, which can comprise the steps of:
a) crossing a first parent plant which can comprise the modified YLS9 gene and/or the modified HsfA2 gene of the invention with a second parent plant to obtain an F1 population;
b) optionally performing one or more rounds of selfing and/or crossing with a plant from the F1 population to obtain a further generation;
c) selecting a plant that can comprise the modified YLS9 gene homozygously, the modified HsfA2 gene homozygously, or both modified genes homozygously as a resistant plant.

The invention also relates to a method for the production of a plant which is resistant to a Begomovirus, in particular ToLCNDV and/or ToLCPMV, said method which can comprise:
a) crossing a first parent plant of the invention which can comprise modified YLS9 gene and/or modified HsfA2 gene with a second parent plant, which is another plant not comprising the modified YLS9 gene or modified HsfA2 gene of the invention;
b) backcrossing the plant resulting from step a) with the second parent plant for at least three generations;
c) selecting from the third or higher backcross population a plant that can comprise at least the modified YLS9 gene and/or modified HsfA2 gene of the first parent plant of step a).

The invention additionally provides for a method of introducing another desired trait into a plant that is resistant to a Begomovirus, in particular ToLCNDV and/or ToLCPMV which can comprise:
a) crossing a plant which can comprise the modified YLS9 gene and/or modified HsfA2 gene of the invention with a second plant that can comprise the other desired trait to produce F1 progeny;
b) optionally selecting in the F1 for a plant that can comprise the resistance and the other desired trait;
c) crossing the optionally selected F1 progeny with one of the parents for at least three generations, to produce backcross progeny;
d) selecting backcross progeny which can comprise the resistance and the other desired trait; and
e) optionally repeating steps c) and d) one or more times in succession to produce selected fourth or higher backcross progeny that can comprise the resistance and the other desired trait.

Optionally, selfing steps are performed after any of the crossing or backcrossing steps in above described methods. Selection of a plant which can comprise the Begomovirus resistance and the other desired trait can alternatively be done following any crossing or selfing step of the method. The other desired trait can be selected from, but is not limited to, the following group: resistance to bacterial, fungal or viral diseases, insect or pest resistance, improved germination, plant size, plant type, improved shelf-life, water stress and heat stress tolerance, and male sterility. The invention includes a plant produced by this method and a fruit obtained therefrom.

TABLE 1
Sequences.

| ID# | Description | Sequence |
|---|---|---|
| SEQ ID NO: 1 | Wild type Genomic | CATAAAATCGACAAAACCAATAAAAATAATTTGAAAA |
| | DNA sequence | CGTGACTCGCAGGTTTTGATAATTCTTTAAAGAAATA |
| | CsYLS9 | AAGATAAATAAATGAGACAAAAATTTAAATAAGGTGT |
| | | CTAAAACCAAAATACTGAAAAACTGATACATAGAAAC |
| | | GGAAACAAAATTGAGTCTCCATATGACATGTCACAGA |
| | | CCCTTATTGTCACTTGGCTTTCCAGATCCTCTACCTCT |
| | | GCCTGAAAGATTAAATACAGAAAAGAGTGAGTATATA |
| | | AAATATATCCAGTAAGGGATCATCTACTGGTCTCGCT |
| | | AGGTGATTTGTTAACTTTCCATTAGAAACATAATAATA |
| | | GTGTTGTGTGTTCAATGGAGCACACCTAGGCGAGTGA |
| | | GATACTACGAACACACCTAATCGTGTGAGCAATCTCG |
| | | TAGGAACACCCTTAGTCGTGCGAGTGATCGTAGATAC |
| | | ACACTGAGTGAGACCATATGAACACCCCTAGTCATGT |
| | | GAGTGATCGTACATACACACTCTTAAACATGTCTGTG |
| | | ATATGTAGGTACACCTCTTATTGTACACAGTCTAAACG |
| | | ATCTCTTAGTAATAATCTTGTAGCAATGATCTCGTAGC |
| | | AAGTCTAAACGATCTCATAGCAAGTTGAAACGATCTT |
| | | GTACCCTCGTACCTAGCCTTAAACAATCTTGTACCCAG |
| | | TCTAAACGATCTTATACCTAGTCTAAACGATCTTTGTA |
| | | TCTAGTTTAAACAATCTTGTACCAAATCAAAACATCTA |
| | | TTAGTGATAGTGGTCTATCTGTCTATCTAGGATAAACA |
| | | ACTGATCGTTTAGACATTGGTACACGATCGTTTAGATC |
| | | TTGTACCAAAAAGAAAAAAAAGAAGATGAGAAATGA |
| | | AGAAAAAGGAAGAAATTCTGGAAGAAGAAATAAAAA |
| | | AATTGGTAAATATTTACATAAATAACCATAATATTTTA |
| | | AAATGAATAAGAAGTAATAATACAAAGAAATTAATA |
| | | ATAAGAAAAAGAGACGAATAAATCGCAAAAAAGAAA |
| | | AAGAAAAAGAAGTGAAAAATCGTCAGCACTATTAAG |
| | | GGTAAATTTGGAATTTATGAAAGTACCATGAGTTTTTT |
| | | AATTTTATTATATGGGTCGTAAATATTTTGGTCATGTT |
| | | TTGTTATATTTATAAAAATTAACCAAACATAATAGTAC |
| | | AGAAAATTATACATGGTGCAGTAAAATAACCTACATT |
| | | TAATTTAGGAAAATTGTATTAAATGACAAAAATATTT |
| | | AGAAAAAAACAGCTCATGACACTTATTTTTTGTATATT |
| | | GTGAATATGACAAAATTAGTGATATCAGATGACTATT |
| | | AGATGATAATCATAGGGCCATCGGAGGGCTATTGACG |
| | | ATAATCATAGAGTTATCGACTTTTAAATTTGCTACTCT |
| | | CGCAATTTAGAAAATGTAATGACATGAGCCTTATTGT |
| | | CATAATTTGGTGGTTCCAGAAAATTGATTGGAAAAGA |
| | | GGAGTAGGAAATGATGAGATCAAGAACCAAACAAGT |
| | | AAGCCATAGAAAGTTCTCCACTTGCTCTGGTGGTTCCA |
| | | TATGCAAGTGGGAAACTTTCTATGGCTTACTCGGTCTT |
| | | GTCATTTTCTGCTCCTCTTTTCTCAATCAATTTTCTGGA |
| | | ACCACTAGAGCAAACTAGAGTTGTTTCTGTTTGAGAC |
| | | AACCTCATATTTCTTTCGACGCAACTCGTAATAATGTT |
| | | CAACTTTCCTCTTTGCACATGCTGCTCATGTCCCTATG |
| | | ATCATACTAGGCAATTATGCTGAGGCAATGCTTTTAGT |
| | | TATTCATGTAATCTTTGAACCTACAAACGTGTTTTTTT |
| | | TTTTAATATTTGACTTAATTTTAATATAATTTTGATTTG |
| | | ATATTTTAATAAATTTTGAATAAATTTTATGTTAATTG |
| | | TTTTTAGAATTTGATTAATTTAAATTGAATTCGATCCC |
| | | AATAGTGAATAAAATTAAATAATAATATATTAAAAAA |
| | | TTATTACAAAAACGTTTTCCTGATGCACAAAGCACGTT |
| | | GGCATGGACACAATTGCTGACGCTTTGACCAGATGTT |
| | | AGACGAGATTTTTCTCGATGTCTCACGTGAGTCAGCG |
| | | AACCCCAATCGGGCAAACGTTTTTGTCGACGTCATCG |
| | | TGTTTGCTGACATCTCGACGCTGTAATGGTGACGTTCT |
| | | TGTCGACGTTAGTTTTGGCGTCGGGATAGATATTCTCG |
| | | ACGTGTTTTTGTGAATTTACTGACACATGTGTGCGTCA |
| | | GATGAACCCTTACTTCTTGTAGTGATAATGTTCTTTTC |
| | | TATTTTAATTTGGTTGAAAAAATACATTTTCCATAATT |
| | | CTTAAAAAAGAATGTCTCTCCATTTTCAAAATCTTGAT |
| | | GTTCGGTTGATCCACAAGGTTTCCATTACAAGAAATT |
| | | GTGTTTTTAGCGGCGCACCAAAAATGTCACTAAAATG |
| | | CAAAATACATCGCTAAAAGTATTAGCGACGCAAAGCC |
| | | ATTTGTCGCTAAATCCATGTCGTTAAAAGTTTTAGCAA |
| | | TAATGCAACAATTACTCACCGCAAATGAGATACTTTT |
| | | AGTGACGTTTTCGTGTTACTTTTAGTAACATAATTTAG |
| | | TAATAAATAAATGTTGTTGTTAAAGGTTAATTTGAAAT |
| | | CATTAATTACAAATATCTTTTGCGACATATTGCAGTGC |
| | | GTCACTAGTAATGCTTTTGTGACGTAATTGAAAATGCC |
| | | AATAAAAATATTTTATTATATAAAAAAATAATATCCA |
| | | ATTTATTATATAAATCTGTTATAAAATGTTAAATATAA |
| | | TAATATGAAAAACTATCACGAGAGAATGTGCAACAAA |
| | | ACCAACTTTATCTCAAAAAGAAATATATGTAAAGAAA |
| | | CTTCAATAAATTCATAAATAGTTCAAGCTTTCACTGAT |
| | | GTATTAAGGATTTTTCATATGCAACTTGTTTGTTTTTCT |
| | | CTTTTTGCCACATGGGCTTTTTATGTATAAACTTGAAC |
| | | ATTATTTTAGCCATTACCCACATCTAAACAAAATAAA |
| | | GCGTACTTAACTCAAATCTTGAACTACAAGTAAACAC |
| | | AAAATACACCCTAATTTTAAATAGTGAGGATATATAC |
| | | CTGTACCCCTTCATTAAGCAAACCAAAATCATATAAT |
| | | ATCCCTTCACTTATACATGAGAGAACAAAGATCAAAA |
| | | CCAAAAAAGAATATATATATATATATATCTATTTTTGA |
| | | AGAACATGAGGAGTACTACTACACAGGGAGAAGGAG |
| | | CATCATCCTCCATTATTGAGGCACCAAAACGAAGCTT |
| | | CTGTAGACAACGTGAGACAACAAAACGCACAAGAAT |
| | | CATAAGAATCATAGGAAGAAGTTTGTTGTCTGTAATA |
| | | ATCTTCTTGAGTGTTGCAATTATCACATGTTGGCTTGT |
| | | TGTTTTCCCCAGAACCCCACGTCTCATGGTGGAAACTA |
| | | GCAAAGTGACAGCCCATGGTTCAACTAATAGACACCT |
| | | CAATGCAACCATAGTTTTCTACATCAAAAGCTACAAC |
| | | CCTAACAAAAAAGCCTCCATTCACATGGATTCTGTGA |
| | | AGATGATAGTCAGTGATTATATGGGGCTACCGTTTCA |
| | | CTCCACCATCCCCACCTTCACGTTGATGCCTCGAAACG |
| | | AGATGGTCTTCAACTCAACCGTTCGTGTCAACTTCATG |
| | | TACCCATTTGGGCGCCCGGTGCATTCGGACTGGGTAC |
| | | ATCTAGAGCTTCGCTTCTCTGCTCAAGTTAGGTACGTA |
| | | CAAACATGTATGAACATAGAAGTGTTGTTGGTTTTGTT |
| | | GGTGTAAATTATTTATTTTTATTCTTACTTTAGAATTA |
| | | GAATTAGTTTTGAAAAACTCTTAAAATTTTAGGAGTAT |
| | | TATTTTTTACACAAAATGGTAACTGTTCTATCATTTTT |
| | | GAATTCTTTTTGACATATAAGAGTATTTTTTAAACATT |
| | | TGAAAGTTGAAATATATTTTTTGACATAAGTAAAAAG |
| | | TTGGAAGAGATATTTTGTATAATTTTGCCTTAATTGCT |
| | | TTTAGATTTTAATTAAAATTAAACCGATGGATACTACT |
| | | AATACCAAAATTTGCCTATATCTTTAATAAGTCATGGC |
| | | TTTGTTTGTATCAGTTACATTGTGAACAGATGGAGGTC |
| | | GAAACCACGATTGCTGGAGATCTATTGTGATCACCTTT |
| | | GGCTTAGGATTAATGATTCTACCCCTAATTTTGATAAA |
| | | ACCAAATGCAGAGTGGATCTTTAAAGAGATTTGATCT |
| | | AGATGTGTTGTTGATCTTGTTTAAGAAGATATGTTATT |
| | | CATGCTGGTTATATAATTCCAAAAGATTTGTAATTGAA |
| | | ATATTCATTTCCTTTAGGTTTAATTTCTTTCTTTATCAA |
| | | TAGAGTTCTTAATGTTTTATAATGTTTTTTTTTAATGGT |
| | | TAAAATATCATTTTCATCAATGTACTTTGATAATTGTT |
| | | CTATTTAGATCTATATATATATTTGTGCTTTAATTCAA |
| | | AATGTCAAATTTTAGTGCCTATAAATTGGATATAAAA |
| | | ATCAAACGTGTTGGCAAAGGAAAACAAAAGTGATAT |
| | | AGGCTATAGAAAAGTCTAACACATCTCTGGCTTTAGA |
| | | AACCATTTTTTTCCTATCCTTTCCTTTCTTCTTTTCTCCT |
| | | CTCCAACTTCATCTTCACTGTGGTTATCGATCATTCTC |
| | | TCCCTTTCTCTTCTTTCTCTATTTTCCCTCTTTATCCCTT |
| | | CGACGCTTGTCGATGTTCACGGCCACCATCTTGTCGCC |
| | | GTCCATAGCTCATCCATTTTGATGAACCCAAATTAGA |
| | | GCTCAATAAATTTAAAGTATGTTTGATCAAAACCGAT |
| | | TCGAAATATCATGTGATGTGTAATATACAAATGAATA |
| | | GAAGGTAAAGGAATTCAACGTTTTGCAAACAAACACC |
| | | CAAATCTATTATTCCATATGATCTGGCTTTTGGTTCCT |
| | | GTAGTTGTGGCCCAATAATTAGTAGCGCGATGTTAAG |
| | | CTATGGACATTGGCGGTGATAGAGCGCATATGTGGTC |
| | | TATAGATGGGAGCAGTTGAACAACAATGCGACCGTGA |
| | | GCAATAGTAAGTATTACATTGGTGTTGTAGCGACCCG |
| | | ACTTCTTATGATTACCAACAAATTAGATATTGTTATAT |
| | | GGATAATATCATAAAGAAATTAGAAAATATTAACGTA |
| | | GAACACGATATATCTATTAGAGTCGGTCTAATGAATT |
| | | CACGTTTTCTCTTTTAAGACAAGTTTATTCATTCTAGG |
| | | GCCGGAATGAGCCGGAGTTTCATGAGTAATTGTCTCT |
| | | TAAGATGAAATAGGATTAACTTTCTCATGTAACAACA |
| | | CCTCGTATACAAAATCCACCGAACTATACTTTGTAAA |
| | | AATAAACCGAACTTGAACAAAGTTTACAAAAGAGAG |
| | | AAATGTCTATGAGAGAGAATATAATTATGAAAAAAAT |
| | | TCTCTTAAAAGCAACTTGAAGGAGTGGCTTATTTAATT |
| | | TATAGACTATTCGTGATCATTCGAATAGTCACATTTCT |
| | | TTTTCAAATTGTTGTTTTTGAAAAAACATATTTATTCA |
| | | TGAAACAATATAACGCTGTATACGAATAGCCATATAC |
| | | CATTTATTICAATAGTTGTGCATGCAAAATTTTTAAAG |
| | | CAGATCATCCGACGAATTTTAGCCACAAAATGAAATT |
| | | AATTCCACAAGGTAAATATCATTCATGAAGAGAGGAT |
| | | TTTAAGGGCTTATTTCATAGAGAAATCTAGACTATATT |
| | | TTATGTTTTCAGATTTATTGAGTTCAACAAATACTACA |
| | | AAAAATTTTGGTCTCAGCCAACAATTTTAATTTTCTGA |
| | | TAACGTAAAAAAATGTGAACAAAGATATGACGTGCCA |
| | | ACGCAATTCCATTACGTGGACGCATAATCGTTTTTGTC |
| | | GACCAAAATATATTTGTGGGTAAAAATGTTGCGCAGT |
| | | CTTCAAAAACTTTTTTAAGTTGGATTTCTATTGACATT |
| | | TATTTTAGTTTATGACCATATTTTAAATATTTGCCGAC |
| | | ATAATTTACGTGCGAAAAACCCCACTTTTAAAAAAAC |
| | | CTATTCTCTTTATTTATTTTAGCACACTTTCTAAACAAC |
| | | CCACATTTTCCCCCTTCTAAAAACCCACATAATTTGTC |
| | | CCTCTTTTTAAGTTATTAGGTTAGGCGATTAGGTTCCT |
| | | TCTGCTTCAAAATCGCCTGACGTCTCTCCATTTGATCC |
| | | ATCATTATCTCCATCCCTCGCCAAGAATAACTCTCTTC |
| | | CTGCAGTTGTTAACTCTCAACCTCGCTCACGAACCATT |
| | | GTCGGTCTCCAATCGTCATTCACCGTTCCCGTCGTCGA |
| | | GCCTCTTTCACCGCCCGTCCAACGTAGATCAAAGTTGC |
| | | CATTGCTGTTGGTGAGTTTCTCGTGAATAATCTCTCTC |
| | | TTTTACCCGCTTTTCCCCCTTATTTTAAATTTCAGTTTA |
| | | TGTTCATATGCAAACTTTTTTGTTTCCTGCATTAATGC |
| | | AATTTTCCCTTCAGTTGGATACATAAGTCTTTTTTTTTA |
| | | CACTTTATGGGTACAAATGTAATTTCAATTATTTATTT |
| | | TGATACTTGATTCATTTGAAACAACAGTAGTTAAGCTC |
| | | TTATTCATCTTAATGTATCTTTTTCAGGTTTGCCTATGA |
| | | TGACTCTATTTTTGTGAAGATTTTCTAAACGGGCTAAC |
| | | TTTTTTTTTTTGTGTGTATTTAATATTTTTTTGTTACCA |
| | | AAATAAAAATTAATATAGCTCATATGACTATACACAC |
| | | TAAAGAATTATGCTTGGTTAAATTTTGACCAAAATATT |
| | | AATGATATATAATATTAATAATAAATGTATAAGAAAT |
| | | TAAAATAATTTTTGTCATGCTTTTTGTGCTACTGGAAA |
| | | TATGTTGGTAGTTAGAAAGCTCATATTGCAGTTTTGTG |
| | | CGTTGGCAAAAAAAAAGACAAATATTATATGCCAATG |
| | | CATCATTTGTGGCCAACAAACAAATTTCTATCCAACA |
| | | AAATTATATGACACATGTATGTATGTATATATATATAT |
| | | ATATATTTCTGCTTACGAGTTTAGTGTGGGAAGAAGTT |
| | | TTCTTTTTGCAGCTGACAAAAATATTTTTGTTGGTAGA |
| | | AGCAATTTCAGCTGACGAAAAAAAATGTAAATATATA |
| | | AATTC |
| SEQ ID NO: 2 | Wild type CDS | ATGAGGAGTACTACTACACAGGGAGAAGGAGCATCA |
| | CsYLS9 | TCCTCCATTATTGAGGCACCAAAACGAAGCTTCTGTA |
| | | GACAACGTGAGACAACAAAACGCACAAGAATCATAA |
| | | GAATCATAGGAAGAAGTTTGTTGTCTGTAATAATCTTC |
| | | TTGAGTGTTGCAATTATCACATGTTGGCTTGTTGTTTT |
| | | CCCCAGAACCCCACGTCTCATGGTGGAAACTAGCAAA |
| | | GTGACAGCCCATGGTTCAACTAATAGACACCTCAATG |
| | | CAACCATAGTTTTCTACATCAAAAGCTACAACCCTAA |
| | | CAAAAAAGCCTCCATTCACATGGATTCTGTGAAGATG |
| | | ATAGTCAGTGATTATATGGGGCTACCGTTTCACTCCAC |
| | | CATCCCCACCTTCACGTTGATGCCTCGAAACGAGATG |
| | | GTCTTCAACTCAACCGTTCGTGTCAACTTCATGTACCC |
| | | ATTTGGGCGCCCGGTGCATTCGGACTGGGTACATCTA |
| | | GAGCTTCGCTTCTCTGCTCAAGTTAGTTACATTGTGAA |
| | | CAGATGGAGGTCGAAACCACGATTGCTGGAGATCTAT |
| | | TGTGATCACCTTTGGCTTAGGATTAATGATTCTACCCC |
| | | TAATTTTGATAAAACCAAATGCAGAGTGGATCTTTAA |
| SEQ ID NO: 3 | Wild type Protein | MRSTTTQGEGASSSIIEAPKRSFCRQRETTKRTRIIRIIGRS |
| | CsYLS9 | LLSVIIFLSVAIITCWLVVFPRTPRLMVETSKVTAHGSTNR |
| | | HLNATIVFYIKSYNPNKKASIHMDSVKMIVSDYMGLPFH |
| | | STIPTFTLMPRNEMVFNSTVRVNFMYPFGRPVHSDWVH |
| | | LELRFSAQVSYIVNRWRSKPRLLEIYCDHLWLRINDSTPN |
| | | FDKTKCRVDL- |
| SEQ ID NO: 4 | Modified CDS | ATGAGGAGTACTACTACACAAGGAGAAGGAGCATCA |
| | CsYLS9 | TCCTCCATTATTGAGGCACCAAAACGAAGCTTCTGTA |
| | | GAAAACGTGAGACAACAAAACGCACAAGAATCATAA |
| | | GAATCATAGGAAGAAGTTTGTTGTCTGTAATAATCTTC |
| | | TTGAGTGTTGCAATTATCACATGTTGGCTGGTTGTTTT |
| | | CCCCAGAACCCCACGTCTCATGGTGGAAACTAGCAAA |
| | | GTGACAGCCCATGGTTCAACTAATAGACACCTCAATG |
| | | CAACCATAGTTTTCTATATCAAAAGCTACAACCCTAA |
| | | CAAAAAAGCCTCCATTCACATGGATTCTGTGAAGATG |
| | | ATAGTCAGTGATTATATGGGGCTACCATTTCACTCCAC |
| | | CATCCCCACCTTCACGTTGATGCCTCGAAATGAGATG |
| | | GTCTTCAACTCAACCGTTCGTGTCAACTTCATGTACCC |
| | | ATTTGGGCGCCCGGTGCACTCGGACTGGGTACATCTA |
| | | GAGCTTCGCTTCTCTGCTCAAGTTAGTTACATTGTGAA |
| | | CAGATGGAGGTCGAAACCACGATTGCTGGGATCTATT |
| | | GTGATCACCTTTGGCTTAGGATTAATGATTCTACCCCT |
| | | AATTTTGATAAAACCAAATGCAGAGTGGATCTTTAAA |
| | | GAGATTTGA |
| SEQ ID NO: 5 | Modified Protein | MRSTTTQGEGASSSIIEAPKRSFCRKRETTKRTRIIRIIGRS |
| | CsYLS9 | LLSVIIFLSVAIITCWLVVFPRTPRLMVETSKVTAHGSTNR |
| | | HLNATIVFYIKSYNPNKKASIHMDSVKMIVSDYMGLPFH |
| | | STIPTFTLMPRNEMVFNSTVRVNFMYPFGRPVHSDWVH |
| | | LELRFSAQVSYIVNRWRSKPRLLGSIVITFGLGLMILPLILI |
| | | KPNAEWIFKEI- |
| SEQ ID NO: 6 | Wild type Genomic | CTCAAGGTAAGCTCGGTGTCATTCTTCGACGATGAAA |
| | DNA sequence | AGACAGAAACCGCTACCTCCCGTTGGTCCCGGGCTAG |
| | CsHSFA2 | GACGAGAGCTGCAAAGGTAAATTCTTGGTATTAGTAT |
| | | GCTTCATTAGTIGTGGTTTAGAACAATAATGATGTTCT |
| | | CTAACTTGTTCAGCTTGGGAAAGGTTTATCAAAGGAT |
| | | GAGAATGCTCAAAAATTAGCTCTTCAACACTGGCTTG |
| | | AAGCAGTAAGTTCATATAGTTCAAACTTTTAGAGGTT |
| | | AGAGAAGAGGATGTTGACCGTTTTATTGAACTTCTTTT |
| | | GGCCAGATTGATCCCCGCCACCGCTATGGACATAACT |
| | | TGCATTTTTACTATGATGTTTGGTTTGATAGCAAGAGC |
| | | ACACAACCTTTCTTCTACTGGTAATTTCCCCAATCACA |
| | | TTGCTGGTATCATTACACCGAAAGCTTTCATTCTTATC |
| | | CTTCAAGCTTCCTTTTAAAAGGTTGGATATTGGTGATG |
| | | GGAAGAGGGTTAATCTTGAGAAGTGCCGTAGATCAGT |
| | | TCTGTATAAGCAATGCATCAAGTACCTTGGACCGGTA |
| | | AGTCGCCTGATTAGATTGTAGTTTCTACTCCATTACGA |
| | | GCAGTAAGCAATTGTTTCTATTCATTTCTGTTGCAGAA |
| | | AGAAAGGGAGGAGTACCTGGTGATTGTGGAGAATGG |
| | | GAGGCTTGTTTACAAGCAAAGCAGAATACCCATCACC |
| | | ACAGTTGAAGATTCCAAGTGGATTTTTGTACTCAGCA |
| | | CGTCAAGAGATCTGTATGTGGGACAGAAGAAGAAAG |
| | | GTCGCTTTCAACACTCCAGCTTCCTGTCTGGAGGAGCT |
| | | ATAACGGCTGCAGGAAGATTAGTTGCCATTGATGGAA |
| | | TTCTCAAGGTGCATAATTGAATGCTTGAGCATATGAG |
| | | TGTTTTGTAGGATAATCATTCTGTAGAGCGTGTAATTC |
| | | ATAGAGGATGATGTTTTCATTTGAATTTTCTTTAGGCT |
| | | ATATGGCCATACAGTGGTCATTACCTCCCAACAGAGA |
| | | ACAATTTTAAGGAGTTCATTAGTTTCCTTGAAGAACAT |
| | | ACAGTGGACTTGACCAACGTTAAGGTAAGAAATTAGA |
| | | TATATAACTACATGGATGTAGTTTTCTATATCAGCTTC |
| | | TTTTCAGATTGCACAACTTGTTTTCTTGAATTTTCTCAA |
| | | TGGGGATTCCTGCGTTATTATACAGAGATGTTCGGTA |
| | | GATGATGACAACTACTCACTTAACAACACGAGCGAGG |
| | | AAACAACGGAAACAACTTCAGAAGACATGGTTGCAG |
| | | ATGATGTTGATTTGGCAGTACCCGTCAAGTTGGTCAC |
| | | AACTAATGAGCGGCAAGAGGATCAGGGTAGCAGCAG |
| | | AGAGGCACCGTTGATAGACATACCAAAGCGCTTGCTG |
| | | TGCAGATGGAGCAGTGGAGTTGGCCCTAGAATAGGGT |
| | | GTGTGAAGGAGTATCCAGCAGAGTTGCAAGCACGAGC |
| | | ACTGGAGCAAGTGAACTTGTCACCAAGACCGTCACCA |
| | | GGTTTCTTCGGAGGCTCGCTTCCAATACCTTCACCACG |
| | | GCCGAGCCCAAAAATCAGGATGTCTCCTAGGCTCTCA |
| | | TATATGGGGATCCCCAGCCCCAGGGTGCCTGTGTGTC |
| | | TGACAGCGCCCATCTAAAGCTTCTGCCTCTTGTGCCCC |
| | | TAACAAAACCTCTGATTTGTCGCATTTTCATGTACATG |
| | | GAACTCATCCTTCTTTCTGCCTGGAATGATTCGAGTAG |
| | | TACATATCTTGTTTCACTGGATCATTGACTTCCCTGGA |
| | | ATAAAGAACCAATATTTTATTTACCAATCCCCTAAATG |
| | | CGAGGGAAAAGGAAAACAATTCTATAGTTCAGGGGC |
| | | CCATGTTATTTTGGCTGCGTGTTCAATCCCAGCAAAGC |
| | | AATAAATGCAAGAGATCTCAAATGTGTGCCTCCGGAG |
| | | CCCTTCCATACAAAATAACTTGTGAGATGCTTTTTCAT |
| | | GCCATGGAAAATTGCAAACAAACCATGAACTTGAAAA |
| | | ACCCCATCCACTGACATTGACCAAAACCTCTCGCCCT |
| | | GTAAGGAGCATTCTAAAGTGAGAAAGAGAGAGCAGC |
| | | TACCTCTAGGTATATTAATTTCCTTTCTCTTGAGAAAA |
| | | CAACCGTGCGGATTTTATCTTCTTCTGCCACCAAAGGA |
| | | TATCCATCAACCAACAGTACAGAAAATCATAAAAGAA |
| | | TTCTACTCAGTTAATGAAGCCTGAATTTCTAAACTTGT |
| | | GTTTGTTCCATTTTACATAGCTGAGCTTAACATCATTA |
| | | AGCTCAAACAAGAACTATCAACCAAGGACCAATAGCC |
| | | TTTCAAGATCTAATACAATTTTATCAGGAATCTATTCC |
| | | CTGAGTTATGATATAGCTGCGTTTTGTTATCCTCCACA |
| | | AAAAGTAGTGAGCATCAAACATACTTTAAGGGAAAA |
| | | GATTTGGCAGACTCGTACATTTCAAATGAATAACAGT |
| | | TATTGGGTTCCAATTAATGAATGGAATAATTAGTCTTA |
| | | CGTTAATGGTAAAGAAATAAGAATATGACTGAATCTT |
| | | CTCGCAGGTTTCCTTTCTCTGAACATGCCTGCAAATAA |
| | | AACCATGGATGAACAAAAAAAAAAACCACAAACCAA |
| | | TTGAACACAACCAAGAATATTAAGTACATAATATTGA |
| | | AAAAGCCAATGAGTTTCAAAGGATATAAGGAAACAG |
| | | GAACAATCTGTGGCTCCCCTGCAACATCCACGGTTCCT |
| | | TCCTACACGAGCCAGCTGAAACCTATTCCACACGAAG |
| | | AGAAGCAAAATGGCAAACATATTAACTACACTGTTAT |
| | | ATCTATGGCTTTGACCGGAGAAACCCCATTTGATCAA |
| | | CGAGTTCCTGCAAGTCCTCAGTCCAATCTAGGGGTTC |
| | | AGCAATCAGATCCTCCACTTCCACATCAATGTCTGATT |
| | | GATTAACAATGATTGTGGGTTCTTCTGGATGTCCAGCC |
| | | ACAAGATCTTCAACCCAAAGGTGACTGAAAATCCCCG |
| | | ATTCTTCGTGGACAGAATGACCCAAGTCGGAAACAGG |
| | | GTCCGCGATCTCAATGCTTGACTCGTCTTCAAAGTTGA |
| | | CTGTTAAGAGCGTCTCTATATCTGGTTCAGAAGTTTCT |
| | | AGCTCTTCTTGTTTAAGAGCTACAGGTACATTCTCATC |
| | | CAGGAGATTCTCTACACTTGGGCTGGCAGTTAGTCTTC |
| | | TTTTTCTTCCAATTTCTACACCTCTCAATTCCCTTCCCT |
| | | GATTACTATTAATAAATTTCTGAATGAATGAAGGATT |
| | | CTTGAGGGCTTTGCTGAGGAATGTCATGATCTGCTTCT |
| | | GTTTACTCTCAGCTTTCTCTAACCTATCCTCCATGGTC |
| | | ATTATTTTATCTCTTGAGCTCTGGTGTTGCTGCCTCAA |
| | | TCTCACTAATTCCGCCATTAACGTGCTTCTGTCCCTTC |
| | | TCAACCTCTCAAGGTCAGCTTCCAGTCCAAATTGCCCT |
| | | AATTCAACACATGTTCCTCCATGGTGCTGAATGCTTTG |
| | | TTGTGAATGTCTTCTCCTCTTTATGGTTCTTAGCAGATT |
| | | CCGTTGTCCTCCCAGAAACCCCTCATTCGCAAACTCCC |
| | | ATCGATCAGGATCGACTTTACGAAAACCCTTAATTGA |
| | | AGAATTTCAACATCAACGAGATTAGGTTATGAAATTT |
| | | TCAAACATACTTCAGAAACAAAAACCCACAACACTGT |
| | | GGACGTTGTATCAATTAAAACTCCAAACTAATTCGTT |
| | | GCGTTCATAATTCCTACCCATTTCAATTAGCTCCATTC |
| | | AAAATCAGTTTAAATTCACATACGTGAAATTATATCTC |
| | | TACCAAAATACTATCAAAATATGAAGAAAATCGATAC |
| | | GTCAACAAGTAAAGAAAACGGAGAGAAAGACAACGA |
| | | ATATCAATTTCAGTGAAAAACTTACATAAGTATTGAG |
| | | TTGACGAATGAAACTAGAGAAGTTGGAGTGTTTGAAG |
| | | TAACGAGGCAGCAAGGTACTAGAGAATTTGTGATAAT |
| | | CCCAAACGATGAAACTATTACGAGCTCTACTCCATGA |
| | | AACAATAGAGTCCGTCAATGGATCTTCGACCATTTCA |
| | | AAGGTCTTGGTCAGAAATGGAGGTGGACCAACGTCGT |
| | | GTAAGCCTTCAATCGGCTGGGGAGTAACGGATGAAGA |
| | | GGAAGAAGAAAAAGAAGAAGAAGACGCGGTAGCGGT |
| | | GGCCACAAGTGATTCCTCGGGTTTCACTTTCAGTTCAT |
| | | CCATTACAGATTTGATCGTAAGCGAAGTTAGTCAAAG |
| | | ATTGAGAAGCTAATGGGCTTGATTAGTTGAAGAAGAA |
| | | ATGAGTTTAGAGATTGATTGATTGATAAATGGATGGA |
| | | AGAAACTGAATTTTAATGGCGTCGGAGTGGAATGTTC |
| | | TAGGACTCAGCAATGTCTTTTTCAGGTGTTTGTCAGAA |
| | | GCTTCTTCCCCCTTGTCCACCTCACCAAAAATATCTAA |
| | | TCGAAACTTCTCGTTTCTTCCAGGTCTTTTTTTTAATAT |
| | | CAGTCCTTTAAAATTTTCATAAACAAAAATATTTATGA |
| | | TTGCCTAACAAAATCAAAATCAAGATGTTGAGGAAGT |
| | | CGGACAGTTTTTCAAAAATATTTTAAGTTTACCTTCCT |
| | | TTCTATCTTTTTTCTTCACCAATTTTTTTTTTTTTTCTTT |
| | | TCTCAATTTGTTACTTGTTTGTTTCTAACGTATCAAAA |
| | | ATGGTAAAAGCTAAACGACAAATATATTATGCATTGG |
| | | GTGTCAATTTATTGAAATACTTTGTATAGTTGGCCTCT |
| | | GAACTCTTTTCGTTTTCAAAATTATTCTACATATAAAT |
| | | ATCTTATCTTTTTGTTATGTTTTAAAAAATATTGTTTAT |
| | | TTTATAAAATTAAGATCGTTTTAGTATAGTAACCAAA |
| | | ATCACATATTTAGTCTTTAAAATTTAAAATTTTGAAAT |
| | | ACTTTGAGTGTCAAAGTAAAATGATGTTTTTCATTCCA |
| | | ACAAAATACTCAAATGCTAAAAGATAGATGACTAAAG |
| | | ATTTTAATAGACATTTTTATACCCATGTTTTGCACAAT |
| | | ATTAAAAAATGGTCCATTCCTTTTAACAAAACCTTAG |
| | | ATTGTCTTCCTTTATTATCAATATTTGAAACTATAAAA |
| | | TAATTTGTTAGGAAATAAGTAAAAAAGAAAGTAAGA |
| | | AATGAGAAATAATGTAATGATTATATTTGCACTAATC |
| | | AATCAATAAAAAAAGTTTAATTTTGTTAAGAAGCATT |
| | | TTTTTAAATAACAAAATAAATTAAAATATCAAAATTTT |
| | | GAATTATGTCACTCATACTGTTATATCATTCTAACACA |
| | | TTAATAGTTATTCGTGATAAAACTTGTTATAGACGGTA |
| | | AATATTTTATAAAATCTATTGTTTTCTAAAATTCTCTTT |
| | | TTCTTAATTATAATAACAATCATTGTTTTTAGTAAATA |
| | | TTAATTTATTAATCAACTTAAACATCATCTCAGGTATA |
| | | ATTGAACAAAGAGCTGAGGCCTCCATTTGTTGGATTG |
| | | TAAATTGGGCTCTAAAAGCCCACAAAAAAGGATTTTC |
| | | CTTTCATGATTGAAATCCAAGGGATGGATGAATTTTA |
| | | ACCTACCAAATAATTGTAGTCTTCATCCTAAACTCATC |
| | | TATTTATATCCATTGGGTCACTTGCAAAAAAAATGGA |
| | | AAAAGTTACCGTCCACATATTTTATTATTTACAAAAGA |
| | | ATCATAAAATAAAATAATTATAAAAATATGAATTTGA |
| | | AATTTTTTTATTTATATTTGCAAAATCACAAACGTTAC |
| | | ATAGACCGCTAAATTAATTTGTCATCCAATACAATTTT |
| | | CCATATCCATTTACATAGGAAGCTTTTAATTTCATCAA |
| | | ATTAATTTAAAATAATTTACCCGTGTTGTTGTAGCTTT |
| | | TTAATTAATGTTTATTGAGCAAATCTTAGAACAATAG |
| | | ACATACAATTTCATGTCGATTCATAAATTTTATATAGC |
| | | TAGACACTATATAATAAACATGTTTTTTTCTTTTTTTTT |
| | | TTTTGTAAACGTAGGACTTGAACTCTTGGACACGACC |
| | | ACAACATTACAACAACATTTTGTAAAGAATAAAGCAT |
| | | TATTTAATATATATTATAATTTGACAACTACAAAATAT |
| | | TATTAAAATATAAAAACCAATGAAAGGGCACATGTCC |
| | | TCACTGGCTCCTACATAAATTTCTTAGTGCTCATGTCG |
| | | ACAAGGCACAGTTCATTGGATGCAAGTAGTTTACTCT |
| | | GCA |
| SEQ ID NO: 7 | Wild type CDS | ATGGATGAACTGAAAGTGAAACCCGAGGAATCACTTG |
| | CsHSFA2 | TGGCCACCGCTACCGCGTCTTCTTCTTCTTTTTCTTCTT |
| | | CCTCTTCATCCGTTACTCCCCAGCCGATTGAAGGCTTA |
| | | CACGACGTTGGTCCACCTCCATTTCTGACCAAGACCTT |
| | | TGAAATGGTCGAAGATCCATTGACGGACTCTATTGTTT |
| | | CATGGAGTAGAGCTCGTAATAGTTTCATCGTTTGGGA |
| | | TTATCACAAATTCTCTAGTACCTTGCTGCCTCGTTACT |
| | | TCAAACACTCCAACTTCTCTAGTTTCATTCGTCAACTC |
| | | AATACTTATGGTTTTCGTAAAGTCGATCCTGATCGATG |
| | | GGAGTTTGCGAATGAGGGGTTTCTGGGAGGACAACGG |
| | | AATCTGCTAAGAACCATAAAGAGGAGAAGACATTCAC |
| | | AACAAAGCATTCAGCACCATGGAGGAACATGTGTTGA |
| | | ATTAGGGCAATTTGGACTGGAAGCTGACCTTGAGAGG |
| | | TTGAGAAGGGACAGAAGCACGTTAATGGCGGAATTA |
| | | GTGAGATTGAGGCAGCAACACCAGAGCTCAAGAGAT |
| | | AAAATAATGACCATGGAGGATAGGTTAGAGAAAGCT |
| | | GAGAGTAAACAGAAGCAGATCATGACATTCCTCAGCA |
| | | AAGCCCTCAAGAATCCTTCATTCATTCAGAAATTTATT |
| | | AATAGTAATCAGGGAAGGGAATTGAGAGGTGTAGAA |
| | | ATTGGAAGAAAAAGAAGACTAACTGCCAGCCCAAGT |
| | | GTAGAGAATCTCCTGGATGAGAATGTACCTGTAGCTC |
| | | TTAAACAAGAAGAGCTAGAAACTTCTGAACCAGATAT |
| | | AGAGACGCTCTTAACAGTCAACTTTGAAGACGAGTCA |
| | | AGCATTGAGATCGCGGACCCTGTTTCCGACTTGGGTC |
| | | ATTCTGTCCACGAAGAATCGGGGATTTTCAGTCACCTT |
| | | TGGGTTGAAGATCTTGTGGCTGGACATCCAGAAGAAC |
| | | CCACAATCATTGTTAATCAATCAGACATTGATGTGGA |
| | | AGTGGAGGATCTGATTGCTGAACCCCTAGATTGGACT |
| | | GAGGACTTGCAGGAACTCGTTGATCAAATGGGGTTTC |
| | | TCCGGTCAAAGCCATAG |
| SEQ ID NO: 8 | Wild type Protein | MDELKVKPEESLVATATASSSSFSSSSSSVTPQPIEGLHD |
| | CsHSFA2 | VGPPPFLTKTFEMVEDPLTDSIVSWSRARNSFIVWDYHK |
| | | FSSTLLPRYFKHSNFSSFIRQLNTYGFRKVDPDRWEFANE |
| | | GFLGGQRNLLRTIKRRRHSQQSIQHHGGTCVELGQFGLE |
| | | ADLERLRRDRSTLMAELVRLRQQHQSSRDKIMTMEDRL |
| | | EKAESKQKQIMTFLSKALKNPSFIQKFINSNQGRELRGVE |
| | | IGRKRRLTASPSVENLLDENVPVALKQEELETSEPDIETL |
| | | LTVNFEDESSIEIADPVSDLGHSVHEESGIFSHLWVEDLV |
| | | AGHPEEPTIIVNOSDIDVEVEDLIAEPLDWTEDLQELVDQ |
| | | MGFLRSKP- |
| SEQ ID NO: 9 | Modified CDS | ATGGATGAACTGAAAGTGAAACCCGAGGAATCACTTG |
| | CsHSFA2 | TGGCCACCGCTACCGCGTCTTCTTCTTTTTCTTCTTCCT |
| | | CTTCATCCGTTACTCCCCAGCCGATTGAAGGCTTACAC |
| | | GACGTTGGTCCACCTCCATTTCTGACCAAGACCTTTGA |
| | | AATGGTCGAAGATCCATTGACGGACTCTATTGTTTCAT |
| | | GGAGTAGAGCTCGTAATAGTTTCATCGTTTGGGATTAT |
| | | CACAAATTCTCTAGTACCTTGCTGCCTCGTTACTTCAA |
| | | ACACTCCAACTTCTCTAGTTTCATTCGTCAACTCAATA |
| | | CTTATGGTTTTCGTAAAGTCGATCCTGATCGATGGGAG |
| | | TTTGCGAATGAGGGGTTTCTGGGAGGACAACGGAATC |
| | | TGCTAAGAACCATAAAGAGGAGAAGACATTCACAAC |
| | | AAAGCATTCAGCACCATGGAGGAACATGTGTTGAATT |
| | | AGGGCAATTTGGACTGGAAGCTGACCTTGAGAGGTTG |
| | | AGAAGGGACAGAAGCACGTTAATGGCGGAATTAGTG |
| | | AGATTGAGGCAGCAACACCAGAGCTCAAGAGAGAAA |
| | | ATAATGACCATGGAGGATAGGTTAGAGAAAGCTGAG |
| | | AGTAAACAGAAGCAGATCATGACATTCCTCAGCAAAG |
| | | CCCTCAAGAATCCTTCATTCATTCAGAAATTTATTAAT |
| | | AGTAATCAGGGAAGGGAATTGAGAGGTGTAGAAATT |
| | | GGAAGAAAAAGAAGACTAACTGCCAGCCCAAGTGTA |
| | | GAGAATCTCCTGGATGAGAATGTACCTGTAGCTCTTA |
| | | AACAAGAGCTAGAAACTTCTGAACCAGATATAGAGAC |
| | | GCTCTTAACAGTCAACTTTGAAGACGAGTCAAGCATT |
| | | GAGATCGCAGACCCTGTTTCCGATTTGGGTCATTCTGT |
| | | CCACGAAGAATCGGGGATTTTCAGTCACCTTTGGGTT |
| | | GAAGATCTTGTGGCTGGACATCCAGAAGAACCCACAA |
| | | TCATTGTTAATCAATCAGACATTGATGTGGAAGTGGA |
| | | GGATCTGATTGCTGAACCCCTAGATTGGACTGAGGAC |
| | | TTGCAGGAACTCGTTGATCAAATGGGGTTTCTCCGGC |
| | | CAAAGCCATAG |
| SEQ ID NO: 10 | Modified Protein | MDELKVKPEESLVATATASSSFSSSSSSVTPQPIEGLHDV |
| | CsHSFA2 | GPPPFLTKTFEMVEDPLTDSIVSWSRARNSFIVWDYHKFS |
| | | STLLPRYFKHSNFSSFIRQLNTYGFRKVDPDRWEFANEG |
| | | FLGGQRNLLRTIKRRRHSQQSIQHHGGTCVELGQFGLEA |
| | | DLERLRRDRSTLMAELVRLRQQHQSSREKIMTMEDRLE |
| | | KAESKQKQIMTFLSKALKNPSFIQKFINSNQGRELRGVEI |
| | | GRKRRLTASPSVENLLDENVPVALKQELETSEPDIETLLT |
| | | VNFEDESSIEIADPVSDLGHSVHEESGIFSHLWVEDLVAG |
| | | HPEEPTIIVNQSDIDVEVEDLIAEPLDWTEDLQELVDQMG |
| | | FLRPKP- |
| SEQ ID NO: 11 | Wild type CDS | ATGAGGAGTACAACTACACAAGAAGAAGAAGCATCA |
| | CmYLS9 | TCCTCCATTGTTGAGGCACCAAAACGAAGCTTCTACA |
| | | GACAACGTCATGAGACAACAAAACGCACAAGAATCA |
| | | TAAGAATCATAGGAAGAAGCTTGTTATGTGTAATCAT |
| | | CTTTTTGAGTGTTGCAATTATCACATGTTGGCTTGTTG |
| | | TTTTCCCCAGAACCCCACGTCTCATGGTGGAAACTAG |
| | | CAAAGTGACAGCCCATGGTTCAACTAATAGAAAGCTC |
| | | AATGCAACCATTGTTTTCTATATCAAAAGCTACAACCC |
| | | TAATAAAAAAGCCTCCATTTACATGGATTCTATGAAG |
| | | ATGATAGTCAAGGATTATATGGGCCTACCATTTCACTC |
| | | CGCCATCCCCACCTTCACGTTGATGCCTCGAAACGAG |
| | | ACGGTCTTCAACTCAACCGTTCGTGTAAACTTGATATA |
| | | CCCATTTGGGCGCCCGGTGCATTCGGACTGGATACAT |
| | | CTAGAGCTTCGCTTCTCTGCTAAAGTTAGGTACGTACA |
| | | AATATGA |
| SEQ ID NO: 12 | Wild type Protein | MRSTTTQEEEASSSIVEAPKRSFYRQRHETTKRTRIIRIIG |
| | CmYLS9 | RSLLCVIIFLSVAIITCWLVVFPRTPRLMVETSKVTAHGST |
| | | NRKLNATIVFYIKSYNPNKKASIYMDSMKMIVKDYMGL |
| | | PFHSAIPTFTLMPRNETVFNSTVRVNLIYPFGRPVHSDWI |
| | | HLELRFSAKVRYVQI |
| SEQ ID NO: 13 | Wild type CDS | ATGGATGAACTGAAAGTGAAACCGGAGGAATCACTTG |
| | CmHSFA2 | TGGCCACCGCCACCGCCACTGCCACCGCGTCTTCTTCT |
| | | TCTTCTTCTTCCTCTTCATCCGTCACTCCCCAGCCGATC |
| | | TCAGGCTTACACGACGTTGGTCCACCTCCATTTCTGAC |
| | | GAAGACCTTTGAAATGGTCGAAGATCCATTGACGGAC |
| | | TCTATTGTTTCCTGGAGTAGAGCTCGTAATAGTTTCAT |
| | | CGTTTGGGATTATCACAAATTCTCTAGTACCTTGCTGC |
| | | CTCGTTACTTCAAACACTCCAATTTCTCTAGCTTCATT |
| | | CGTCAACTCAATACTTATGGTTTTCGTAAAGTCGATCC |
| | | TGATCGATGGGAGTTTGCGAATGAGGGGTTTCTGGGC |
| | | GGACAGCGGAATCTGCTAAGAACCATAAAGAGGAGA |
| | | AGACATTCACACCAAAGCATTCAGCACCATGGAGGAA |
| | | CATGTGTTGAATTAGGGCAATTTGGACTGGAAGCTGA |
| | | CCTTGAGAGGTTGAGAAGGGACAGAAGCACGTTAATG |
| | | GCGGAATTAGTGAGATTGAGGCAGCAACACCAAAGCT |
| | | CAAGAGAGCAAATAATGGCCATGGAGGATAGGTTAG |
| | | AGAAAGCAGAGAGTAAACAGAAGCAGATCATGACGT |
| | | TCCTCAGCAAAGCCCTCAAGAATCCTTCATTCGTTCAG |
| | | AAATTTATTAATAGTAATCAGGGAAGGGAATTGAGAG |
| | | GCGTAGAAATTGGAAGAAAGAGAAGACTAACTGCCA |
| | | GTCCAAGTGTAGAGAATCTCCAGGATGAGAATGTACC |
| | | AGTAGCTGTTAAACAAGAAGAGCTAGAAACTTCTGAA |
| | | CCAGATATAGAGACGCTCTTAACAGTCAACTTTGAAG |
| | | ACGAGTCAAGCATTGAGATCGCGGACCCTGTTTCCGA |
| | | CATGGGTCATTCTGTCCACGAGGAATCGGGGATCTTC |
| | | AGTCAATTTTGGGCTGAAGATTTTGTAGCTGTCCATCC |
| | | AGAAGAACCCACAATCGTTGTTAATCAATCAGACATT |
| | | GATGTGGAAGTGGAGGATCTGATTGCTGAACCCCCAG |
| | | ATTGGACTGAGGACTTGCAGGAACTTGTCGATCAAAT |
| | | GGGGATTCTCCGATCGAAGCCGTAG |
| SEQ ID NO: 14 | Wild type Protein | MDELKVKPEESLVATATATATASSSSSSSSSSVTPQPISGL |
| | CmHSFA2 | HDVGPPPFLTKTFEMVEDPLTDSIVSWSRARNSFIVWDY |
| | | HKFSSTLLPRYFKHSNFSSFIRQLNTYGFRKVDPDRWEFA |
| | | NEGFLGGQRNLLRTIKRRRHSHQSIQHHGGTCVELGQFG |
| | | LEADLERLRRDRSTLMAELVRLRQQHQSSREQIMAMED |
| | | RLEKAESKQKQIMTFLSKALKNPSFVQKFINSNQGRELR |
| | | GVEIGRKRRLTASPSVENLQDENVPVAVKQEELETSEPDI |
| | | ETLLTVNFEDESSIEIADPVSDMGHSVHEESGIFSQFWAE |
| | | DFVAVHPEEPTIVVNQSDIDVEVEDLIAEPPDWTEDLQEL |
| | | VDQMGILRSKP |

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1a: ToLCNDV Bio Assay

In the process of identifying new sources of ToLCNDV resistance, cucumber plants of GBN1489 (internal breeding accession), and susceptible control Pradera RZ were subjected to a ToLCNDV disease test. Young plants of each of the genotypes were mechanically inoculated with a ToLCNDV isolate that was initially obtained from an infected field in Almeria, Spain, and multiplied in Cucurbita pepo plants. Mechanical inoculation of ToLCNDV was performed using the method adapted from Lopez et al. 2015, such that the ToLCNDV inoculum was prepared using buffer (i) as described (Euphytica. 2015 (204): 679-691). The ToLCNDV disease test was performed in a greenhouse with a daytime/night time temperature regime of 20° C./18° C.

Five young plants of each genotype were mechanically inoculated twice, at 7 and 9 days after sowing. A final assessment was performed 24 days post sowing, by visual scoring of ToLCNDV symptoms, based on the scale described in Table 2. A plant having a disease score of 1-2 according to Table 2, is completely resistant to ToLCNDV. A plant having a disease score of 3-4 according to Table 2, is intermediate resistant to ToLCNDV. A plant having a disease score of 5 and higher according to Table 2, is considered as being susceptible to ToLCNDV.

### Example 1b: ToLCPMV Bio Assay

In the process of identifying new sources of ToLCPMV resistance, cucumber plants of GBN1489 (internal breeding accession), and susceptible control Pradera RZ were subjected to a ToLCPMV disease test. Young plants of each of the genotypes were mechanically inoculated with a ToLCPMV isolate that was collected on cucumber near Yazd, Iran, and multiplied in Cucurbita pepo plants. Mechanical inoculation of ToLCPMV was also performed using the method adapted from Lopez et al. 2015, such that the ToLCPMV inoculum was prepared using buffer (i) as described (Euphytica. 2015 (204): 679-691). The ToLCPMV disease test was performed in a greenhouse with a daytime/night time temperature regime of 20° C./18° C.

Five young plants of each genotype were mechanically inoculated twice, at 7 and 9 days after sowing. A final assessment was performed 24 days post sowing, by visual scoring of ToLCPMV symptoms, based on the scale described in Table 2. A plant having a disease score of 1-2 according to Table 2, is completely resistant to ToLCPMV. A plant having a disease score of 3-4 according to Table 2, is intermediate resistant to ToLCPMV. A plant having a disease score of 5 and higher according to Table 2, is considered as being susceptible to ToLCPMV.

TABLE 2
ToLCNDV and ToLCPMV Plant Disease Test

| Disease | | |
|---|---|---|
| Score | ToLCNDV/ToLCPMV symptoms on plants | |
| 1 | No symptoms; healthy plant | Complete resistance |
| 2 | Some non-specific yellowing due to aging, maturation or | Complete resistance |
| | yellowing not related to viral infection | |
| 3 | No leaf deformation, symptoms starting to develop; | Intermediate resistance |
| | mainly on older leaves, some yellowing spots occur on | |
| | less than 25% of the plant surface; re-growth and the top | |
| | of the plant is symptomless | |
| 4 | No leaf deformation, yellowing symptoms, 25-50% of the | Intermediate resistance |
| | plant affected; yellow spots are more abundant than score | |
| | 3; re-growth and the top of the plant is symptomless | |
| 5 | No leaf deformation, severe yellowing symptoms; up to | Susceptible |
| | 100% of the plant affected with yellow spots and areas | |
| | where yellow spots have merged in the larger yellow | |
| | areas; symptoms are progressive even in newly formed | |
| | leaves | |
| 6 | Yellowing symptoms and some mild leaf deformation | Susceptible |
| | symptoms occur; some shoots and younger leaves show | |
| | some deformed parts; some minor mottling in restricted | |
| | areas | |
| 7 | Severe yellowing; strong deformation and mottling in | Susceptible |
| | older leaves; in the younger parts, emerging shoots and | |
| | newly formed leaves show some milder deformation; up | |
| | to 75% of the plant surface shows deformation; plant is | |
| | still growing | |
| 8 | Severe leaf deformation, entire plant affected; the plant | Susceptible |
| | starts producing micro leaves and will no longer grow | |
| 9 | Extreme severe leaf deformation, entire plant affected. | Susceptible |
| | Plants are dwarfed, necrotic or even die | |

### Example 2: Identification of Candidate Genes

Both candidate genes originate from a QTL mapping, based on 2 F2-populations having different susceptible parental lines (KK5.779 and KK5.755). The common resistant parent is GBN1489. The population that was used for the QTL study contained 209 individuals, of which 144 are from KK5.779*GBN1489 and 65 from KK5.755*GBN1489. The trait was scored in both visual scores and qRT-PCR. Two genetic maps were constructed, for KK5.779*GBN1489 146 polymorphic markers were used, having a maximum spacing of 15 cM. The second map for KK5.755*GBN1489 was mad by using 147 markers, where the maximum gap was 25 cM. In the QTL analysis, 4 QTLs were found, two of which were further investigated.

YLS9—QTL chromosome 1. The QTL found on chromosome 1 was about 44 cM. After several finemapping rounds, this area was reduced to only 0.16 cM and ±65 kb. Within this area the candidate genes that were present, the mutations that link to the phenotypes and the effect of the mutations were investigated. Based on this combination, we found a frameshift of 1 bp severely changing the functionality of the YLS9 gene. The frame shifts causes the encoded protein to acquire an extra transmembrane helix compared to the wild type YLS9 protein. Due to the frame-shift mutation, the protein has also lost two predicted protein-protein interaction sites and two protein-DNA interaction sites. At the same time, the secondary structures are also looking different, along with the exposed and buried parts of the protein. Overall, these changes caused by the frameshift mutation have a severe impact on the final 3D structure.

Next to that, an amino acid substitution at the beginning of the gene was identified (See Table 3).

HsfA2 gen—QTL chromosome 2. The second QTL initially had a size of approximately 86 cM. By finemapping using recombinants we were able to narrow down the area to 5.25 cM and ±2.4 Mb (CS01737 to CS09764). Within this area, the HsfA2 gene proved to be an interesting candidate gene because it comprises several mutations compared to the wild type. It was found that the same mutations were also present in resequenced internal breeding lines other than GBN1489. Disease tests performed on plants of these internal breeding lines showed that these plants exhibit at least intermediate resistance to ToLCNDV. Within this HsfA2 gene, four mutations were found that lead to changes in the encoded protein (See Table 3). Especially the amino acid substitution on position 362 of SEQ ID NO: 8 seems to severely affects the functionality of the protein. Such a substitution will severely reduce side chain flexibility, and while Serine could interact with other biomolecules with potentially three hydrogen bonds and other van der Waals bonds, Proline can only interact with van der Waals bonds.

TABLE 3
Mutations in YLS9 and HsfA2

| | Position of | | | Position of | | |
|---|---|---|---|---|---|---|
| | the mutation | | | the mutation | | |
| | in the WT | Wild | | in YLS | | |
| Chromosome | CDS of | type | Mutant | protein | Amino acid | Type of |
| number¹ | YLS9² | allele | allele | sequence³ | change | mutation |
| 1 | 76 | C | A | 26 | Q > K | Amino acid |
| | | | | | | substitution |
| 1 | 551 | A | Deletion | 184 | E > frameshift | Frame shift |
| | | | | | | due to deletion |
| | Position of | | | Position of | | |
| | the mutation | Wild | | the mutation | | |
| | in the WT | type | Mutant | in HsfA2 | | |
| Chromosome | CDS of | (SNP) | (SNP) | protein | Amino acid | Type of |
| number¹ | HsfA2⁴ | allele | allele | sequence⁵ | change | mutation |
| 2 | 65-67 | CTT | Deletion | 22 | Deletion of S | Amino acid |
| | | | | | | deletion |
| 2 | 561 | T | G | 187 | D > E | Amino acid |
| | | | | | | substitution |
| 2 | 792-794 | AGA | Deletion | 265 | Deletion of E | Amino acid |
| | | | | | | deletion |
| 2 | 1084 | T | C | 362 | S > P | Amino acid |
| | | | | | | substitution |

¹Based on the publicly available genome assembly of Cucumis sativus L. var. sativus cv. 9930 version 3, Qing Li et al. (2019).
²Position based on Cucumis sativus YLS9 wildtype CDS sequence (SEQ ID NO: 2).
³Position based on Cucumis sativus YLS9 wildtype protein sequence (SEQ ID NO: 3).
⁴Position based on Cucumis sativus HsfA2 wildtype CDS sequence (SEQ ID NO: 7).
⁵Position based on Cucumis sativus HsfA2 wildtype protein sequence (SEQ ID NO: 8).

### Example 3: Combining a Modified YLS9 and a Modified HsfA2 Gene into One Plant

From the fine mapping populations as described in Example 2 three plants were selected that carried the modified YLS9 gene and the wild type HsfA2 gene, both homozygously. These plants were selfed and the resulting seed comprising the modified YLS9 gene was deposited with the NCIMB under accession number NCIMB 43586.

In a similar fashion two plants were created carrying only the modified HsfA2 gene homozygously, seed of which was deposited under accession number NCIMB 43587.

Plants grown from seed as deposited under accession number NCIMB 43586 were subjected to a disease test as described in Example 1. All plants scored at least intermediate resistant and on average these plants scored 3.6, which shows these plants exhibit intermediate resistance to TolCNDV.

Similarly, plants grown from seed as deposited under accession number NCIMB 43587 were subjected to a disease test as described in Example 1. All plants scored at least intermediate resistant and on average these plants scored 2.8, which shows also these plants exhibit intermediate resistance to TolCNDV.

In order to produce a plant that shows complete resistance to ToLCNDV, a plant of deposit NCIMB 43586 was crossed with a plant of deposit NCIMB 43587. The resulting plant from the F1 was selfed in order to obtain an F2 population.

Plants of the F2 population were sampled for DNA, which was used for a marker analysis using a marker based on the frameshift mutation on position 184 of SEQ ID NO: 2 (See Table 3) and using a marker based on the substitution on position 1084 of SEQ ID NO: 8 (See Table 3). The plants of the F2 population were also subjected to the disease test as described in Example 1.

Plants that were susceptible to ToLCNDV in this test had a marker profile that indicated that those plants either carried the wild type YLS9 and HsfA2 genes homozygously, or that the plants were heterozygous for one or both of the genes, while the other one was homozygous for the wild type.

Plants of the F2 population that scored as intermediate resistant were according to the marker profile homozygous for one of the modified genes, while the other gene was either present in heterozygous form, or was present homozygously in wild type form.

Finally, the marker analysis showed that only plants that scored completely resistant in the disease test were homozygous for both modified genes of the invention.

### Example 4: ToLCPMV Resistance with Only a Modified YLS9 or Modified HsfA2 Gene in a Plant

As described above the seeds of deposit NCIMB 43586 comprise the modified YLS9 gene homozygously. Plants grown from seed as deposited under accession number NCIMB 43586 were subjected to a disease test as described in Example 1b. All plants scored either 1 or 2 according to the scale described in Table 2. This shows that plants comprising only the modified YLS9 gene homozygously exhibit complete resistance to TolCPMV.

Similarly, plants grown from seed as deposited under accession number NCIMB 43587 were subjected to a disease test as described in Example 1b. All plants scored either 1 or 2 according to scale described in table 2. This shows that plants comprising only a modified HsfA2 gene homozygously exhibit complete resistance to TolCPMV.

The invention is further described by the following numbered paragraphs:
1. A modified YLS9 gene, the wild type of which has a coding sequence according to SEQ ID NO: 2 encoding a protein having SEQ ID NO: 3 or the wild type of which encodes a protein having at least 70% sequence identity to SEQ ID NO: 3, wherein the modified YLS9 gene comprises one or more nucleotides replaced, inserted and/or deleted relative to the wild type, and wherein said one or more replaced, inserted and/or deleted nucleotides result in an absence of functional YLS9 protein.
2. The modified YLS9 gene of paragraph 1, wherein the modified YLS9 gene confers at least intermediate resistance against a Begomovirus, in particular ToLCNDV and/or ToLCPMV, when homozygously present in a plant.
3. The modified gene of paragraph 1 or 2, wherein the modified gene comprises one or both of the following mutations:
a) a deletion of an adenine on position 551 in SEQ ID NO: 2, or on a corresponding position of a homologous sequence having at least 70% sequence identity to SEQ ID NO: 2;
b) a nucleotide substitution on position 76 of SEQ ID NO: 2, wherein the cytosine is replaced by a adenine, or on a corresponding position of a homologous sequence having at least 70% sequence identity to SEQ ID NO: 2, or wherein the modified gene encodes a protein having an amino acid replacement on position 26 of SEQ ID NO: 3 or on a corresponding position of a homologous amino acid sequence having at least 70% sequence identity to SEQ ID NO: 3.
4. The modified gene of paragraph 1 or 2 wherein the modified gene comprises a premature stop codon that leads to an absence of functional YLS9 protein.
5. A modified HsfA2 gene, the wild type of which has a coding sequence according to SEQ ID NO: 7 encoding a protein having SEQ ID NO: 8 or the wild type of which encodes a protein having at least 70% sequence identity to SEQ ID NO: 8, wherein the modified HsfA2 gene comprises one or more nucleotides replaced, inserted and/or deleted relative to the wild type, and wherein said one or more replaced, inserted and/or deleted nucleotides result in an absence of functional HsfA2 protein.
6. The modified HsfA2 gene of paragraph 5, wherein the modified HsfA2 gene confers at least intermediate resistance against a Begomovirus, in particular ToLCNDV and/or ToLCPMV, when homozygously present in a plant.
7. The modified gene of paragraph 5 or 6, wherein the modified gene comprises one of the following mutations or any combination thereof:
a) a nucleotide substitution on position 1084 in SEQ ID NO: 7 wherein a thymine is replaced by a cytosine, or on a corresponding position of a homologous sequence having at least 70% sequence identity to SEQ ID NO: 7, or wherein the modified gene encodes a protein comprising an amino acid replacement of Serine to Proline on position 362 of SEQ ID NO: 8 or on a corresponding position of a homologous amino acid sequence having at least 70% sequence identity to SEQ ID NO: 8;
b) a deletion of a triplet encoding a Glutamic acid on position 265 in SEQ ID NO: 8, or on a corresponding position of a homologous sequence having at least 70% sequence identity to SEQ ID NO: 8;
c) a nucleotide substitution on position 561 in SEQ ID NO: 7 where a thymine is replaced by a guanine, or on a corresponding position of a homologous sequence having at least 70% sequence identity to SEQ ID NO: 7, or wherein the modified gene encodes a protein comprising an amino acid replacement of Aspartic acid to Glutamic acid on position 187 of SEQ ID NO: 8 or on a corresponding position of a homologous amino acid sequence having at least 70% sequence identity to SEQ ID NO: 8;
d) a deletion of a triplet encoding a Serine on position 22 in SEQ ID NO: 8, or on a corresponding position of a homologous sequence having at least 70% sequence identity to SEQ ID NO: 8.
8. A plant comprising the modified YLS9 gene of any one of the paragraphs 1 to 4.
9. A plant comprising the modified HsfA2 gene of anyone of the paragraphs 5 to 7.
10. The plant of paragraph 8 or 9 wherein the modified YLS9 gene or the modified HsfA2 gene is present homozygously and wherein the plant exhibits at least intermediate resistance a Begomovirus, in particular ToLCNDV and/or ToLCPMV.
11. A plant comprising the modified YLS9 gene of any one of the paragraphs 1 to 4 and the modified HsfA2 gene of any one of the paragraphs 5 to 7.
12. The plant of paragraph 11, wherein at least one of the modified genes, preferably both modified genes are present homozygously and wherein the plant exhibits at least intermediate resistance to a Begomovirus, in particular ToLCNDV and/or ToLCPMV.
13. A plant of any one of the paragraphs 8 to 12, wherein the plant belongs to the Cucurbitaceae plant family.
14. The plant of any one of the paragraphs 8 to 13, wherein the plant is a Cucumis sativus plant or a Cucumis melo plant.
15. The plant of any one of the paragraphs 8 to 14, wherein the plant is an agronomically elite plant, in particular a hybrid variety or an inbred line.
16. A seed capable of growing into a plant of any one of the paragraphs 8 to 15.
17. A fruit harvested from the plant of any one of the paragraphs 8 to 15, wherein the fruit comprises the modified gene of any one of the paragraphs 1 to 4 and/or the modified gene of any one of the paragraphs 5 to 7.
18. Propagation material suitable for producing a plant of any one of the paragraphs 8 to 15, wherein the propagation material is suitable for sexual reproduction, and is in particular selected from a microspore, pollen, ovary, ovule, embryo sac and egg cell, or is suitable for vegetative reproduction, and is in particular selected from a cutting, root, stem cell, and protoplast, or is suitable for tissue culture of regenerable cells or protoplasts, which regenerable cells or protoplasts are in particular selected from a leaf, pollen, embryo, cotyledon, hypocotyl, meristematic cell, root, root tip, anther, flower and stem, and wherein the propagation material comprises the modified YLS9 gene of any one of the paragraphs 1 to 4 and/or the modified HsfA2 gene of any one of the paragraphs 5 to 7.
19. Use of the modified YLS9 gene of any one of the paragraphs 1 to 4 and/or the modified HsfA2 gene of any one of the paragraphs 5 to 7 for producing a plant that is resistant against a Begomovirus, in particular against ToLCNDV and/or ToLCPMV.
20. A marker for the identification of a modified YLS9 gene of any one of the paragraphs 1 to 4, wherein the marker detects a substitution from a cytosine to an adenine on position 76 of the wild type YLS9 gene sequence of SEQ ID NO: 2, or wherein the marker detects a deletion of an adenine on position 551 of the wild type YLS9 gene sequence of SEQ ID NO: 2, or wherein the marker detects the said substitution or said deletion on a corresponding position of a homologous sequence that has 70% sequence identity to SEQ ID NO: 2
21. A marker for the identification of a modified HsfA2 gene of any one of the paragraphs 5 to 7, wherein the marker detects a deletion of a triplet CTT on position 65-67 of the wild type HsfA2 gene sequence of SEQ ID NO: 7, or wherein the marker detects a deletion of a triplet AGA on position 792-794 of the wild type HsfA2 gene sequence of SEQ ID NO: 7, or wherein the marker detects a substitution from a thymine to a guanine on position 561 of the wild type HsfA2 gene sequence of SEQ ID NO: 7, or wherein the marker detects a substitution from a thymine to a cytosine on position 1084 of the wild type HsfA2 gene sequence of SEQ ID NO: 7, or wherein the marker detects any of the said substitutions or said deletions on a corresponding position of a homologous sequence that has 70% sequence identity to SEQ ID NO: 7.
22. Use of the marker of paragraph 20 and/or paragraph 21 for identification of Begomovirus resistance, in particular ToLCNDV and/or ToLCPMV resistance.
23. A method for selecting a plant resistant to a Begomovirus, in particular TOLCNDV and/or ToLCPMV, comprising identifying the presence of a modification in the YLS9 gene and/or HsfA2 gene that results in an absence of functional protein, optionally testing the plant for resistance against a Begomovirus, in particular ToLCNDV and/or ToLCPMV, and selecting a plant comprising a modification in one or both genes and is at least intermediate resistance in the optional disease test as a Begomovirus resistant plant, in particular a ToLCNDV and/or ToLCPMV resistant plant.
24. A method for producing a plant resistant to a Begomovirus, in particular ToLCNDV and/or ToLCPMV, comprising the step of introducing a mutation in the YLS9 gene and/or HsfA2 gene by random or site-directed mutagenesis, wherein the mutation results in an absence of functional YLS9 protein and/or HsFA2 protein in said plant.
25. A method for producing a plant exhibiting resistance against a Begomovirus, in particular ToLCNDV and/or ToLCPMV comprising the steps of:
a) crossing a first parent plant comprising the modified YLS9 gene of any one of the paragraphs 1 to 4 and/or the modified HsfA2 gene of any one of the paragraphs 5 to 7 with a second parent plant to obtain an F1 population;
b) optionally performing one or more rounds of selfing and/or crossing with a plant from the F1 population to obtain a further generation;
c) selecting a plant that comprises the modified YLS9 gene homozygously, the modified HsfA2 gene homozygously, or both modified genes homozygously as a resistant plant.
26. A method for producing hybrid seed resistant to a Begomovirus, in particular ToLCNDV and/or ToLCPMV, comprising the steps of
crossing a first parent plant with a second parent plant, wherein both parent plants are homozygous for the modified YLS9 gene of any one of the paragraphs 1 to 4 and/or the modified HsfA2 gene of any one of the paragraphs 5 to 7, and harvesting the hybrid seed.
27. The hybrid seed produced by the method of paragraph 26.
28. A plant grown from the hybrid seed of paragraph 27.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A modified YLS9 gene, the wild type of which has a coding sequence according to SEQ ID NO: 2 encoding a protein having SEQ ID NO: 3 or the wild type of which encodes a protein having at least 70% sequence identity to SEQ ID NO: 3,
wherein the modified YLS9 gene comprises one or more nucleotides replaced, inserted and/or deleted relative to the wild type, and
wherein said one or more replaced, inserted and/or deleted nucleotides result in an absence of functional YLS9 protein.

2. The modified YLS9 gene of claim 1, wherein the modified YLS9 gene confers at least intermediate resistance against a Begomovirus when homozygously present in a plant.

3. The modified gene of claim 2, wherein the Begomovirus is ToLCNDV or ToLCPMV.

4. The modified gene of claim 1, wherein the modified gene comprises one or both of the following mutations:
a) a deletion of an adenine on position 551 in SEQ ID NO: 2, or on a corresponding position of a homologous sequence having at least 70% sequence identity to SEQ ID NO: 2;
b) a nucleotide substitution on position 76 of SEQ ID NO: 2, wherein the cytosine is replaced by a adenine, or on a corresponding position of a homologous sequence having at least 70% sequence identity to SEQ ID NO: 2, or wherein the modified gene encodes a protein having an amino acid replacement on position 26 of SEQ ID NO: 3 or on a corresponding position of a homologous amino acid sequence having at least 70% sequence identity to SEQ ID NO: 3.

5. The modified gene of claim 1, wherein the modified gene comprises a premature stop codon that leads to an absence of functional YLS9 protein.

6. A modified HsfA2 gene, the wild type of which has a coding sequence according to SEQ ID NO: 7 encoding a protein having SEQ ID NO: 8 or the wild type of which encodes a protein having at least 70% sequence identity to SEQ ID NO: 8,
wherein the modified HsfA2 gene comprises one or more nucleotides replaced, inserted and/or deleted relative to the wild type, and
wherein said one or more replaced, inserted and/or deleted nucleotides result in an absence of functional HsfA2 protein.

7. The modified HsfA2 gene of claim 6, wherein the modified HsfA2 gene confers at least intermediate resistance against a Begomovirus, in particular ToLCNDV and/or ToLCPMV, when homozygously present in a plant.

8. The modified gene of claim 6, wherein the modified gene comprises one of the following mutations or any combination thereof:
a) a nucleotide substitution on position 1084 in SEQ ID NO: 7 wherein a thymine is replaced by a cytosine, or on a corresponding position of a homologous sequence having at least 70% sequence identity to SEQ ID NO: 7, or wherein the modified gene encodes a protein comprising an amino acid replacement of Serine to Proline on position 362 of SEQ ID NO: 8 or on a corresponding position of a homologous amino acid sequence having at least 70% sequence identity to SEQ ID NO: 8;
b) a deletion of a triplet encoding a Glutamic acid on position 265 in SEQ ID NO: 8, or on a corresponding position of a homologous sequence having at least 70% sequence identity to SEQ ID NO: 8;
c) a nucleotide substitution on position 561 in SEQ ID NO: 7 where a thymine is replaced by a guanine, or on a corresponding position of a homologous sequence having at least 70% sequence identity to SEQ ID NO: 7, or wherein the modified gene encodes a protein comprising an amino acid replacement of Aspartic acid to Glutamic acid on position 187 of SEQ ID NO: 8 or on a corresponding position of a homologous amino acid sequence having at least 70% sequence identity to SEQ ID NO: 8; or
d) a deletion of a triplet encoding a Serine on position 22 in SEQ ID NO: 8, or on a corresponding position of a homologous sequence having at least 70% sequence identity to SEQ ID NO: 8.

9. A plant comprising the modified YLS9 gene of claim 1.

10. A plant comprising the modified HsfA2 gene of claim 6.

11. The plant of claim 9 wherein the modified YLS9 gene is present homozygously and wherein the plant exhibits at least intermediate resistance a Begomovirus, in particular ToLCNDV and/or ToLCPMV.

12. The plant of claim 10 wherein the HsfA2 gene is present homozygously and wherein the plant exhibits at least intermediate resistance a Begomovirus, in particular ToLCNDV and/or ToLCPMV.

13. A plant comprising the modified YLS9 gene of claim 1 and the modified HsfA2 gene of claim 6.

14. The plant of claim 13, wherein at least one of the modified genes is present homozygously and wherein the plant exhibits at least intermediate resistance to a Begomovirus, in particular ToLCNDV and/or ToLCPMV.

15. The plant of claim 9 or 10, wherein the plant belongs to the Cucurbitaceae plant family.

16. The plant of claim 9 or 10, wherein the plant is a Cucumis sativus plant or a Cucumis melo plant.

17. The plant of claim 9 or 10, wherein the plant is an agronomically elite plant, in particular a hybrid variety or an inbred line.

18. The plant of claim 17, wherein the plant is a hybrid variety or an inbred line.

19. A seed capable of growing into the plant of claim 9 or 10.

20. A fruit harvested from the plant of claim 9 or 10, wherein the fruit comprises the modified gene.

21. A propagation material suitable for producing a plant as claimed in claim 9 or 10,
wherein the propagation material is suitable for sexual reproduction, vegetative reproduction, or a tissue culture of regenerable cells or protoplasts, and
wherein the propagation material comprises the modified YLS9 gene and/or the modified HsfA2 gene.

22. The propagation material of claim 21, wherein the propagation material suitable for sexual reproduction is a microspore, pollen, ovary, ovule, embryo sac or an egg cell.

23. The propagation material of claim 21, wherein the propagation material suitable for vegetative reproduction is a cutting, root, stem cell, or a protoplast.

24. The propagation material of claim 21, wherein the propagation material suitable for tissue culture of regenerable cells or protoplasts is a leaf, pollen, embryo, cotyledon, hypocotyl, meristematic cell, root, root tip, anther, flower or a stem.

25. A marker for the identification of the modified YLS9 gene of claim 1,
wherein the marker detects a substitution from a cytosine to an adenine on position 76 of the wild type YLS9 gene sequence of SEQ ID NO: 2, or
wherein the marker detects a deletion of an adenine on position 551 of the wild type YLS9 gene sequence of SEQ ID NO: 2, or
wherein the marker detects the said substitution or said deletion on a corresponding position of a homologous sequence that has 70% sequence identity to SEQ ID NO: 2.

26. A marker for the identification of the modified HsfA2 gene of claim 6,
wherein the marker detects a deletion of a triplet CTT on position 65-67 of the wild type HsfA2 gene sequence of SEQ ID NO: 7, or
wherein the marker detects a deletion of a triplet AGA on position 792-794 of the wild type HsfA2 gene sequence of SEQ ID NO: 7, or
wherein the marker detects a substitution from a thymine to a guanine on position 561 of the wild type HsfA2 gene sequence of SEQ ID NO: 7, or
wherein the marker detects a substitution from a thymine to a cytosine on position 1084 of the wild type HsfA2 gene sequence of SEQ ID NO: 7, or
wherein the marker detects any of the said substitutions or said deletions on a corresponding position of a homologous sequence that has 70% sequence identity to SEQ ID NO: 7.

27. A method for selecting a plant resistant to a Begomovirus comprising
identifying the presence of a modification in the YLS9 gene and/or HsfA2 gene that results in an absence of functional protein,
optionally testing the plant for resistance against a Begomovirus, in particular ToLCNDV and/or ToLCPMV, and
selecting a plant comprising a modification in one or both genes and is at least intermediate resistance in the optional disease test as a Begomovirus resistant plant resistant plant.

28. A method for producing a plant resistant to a Begomovirus comprising
introducing a mutation in the YLS9 gene and/or HsfA2 gene by random or site-directed mutagenesis,
wherein the mutation results in an absence of functional YLS9 protein and/or HsFA2 protein in said plant.

29. A method for producing a plant exhibiting resistance against a Begomovirus comprising:
a) crossing a first parent plant comprising the modified YLS9 gene of claim 1 and/or the modified HsfA2 gene of claim 6 with a second parent plant to obtain an F1 population;
b) optionally performing one or more rounds of selfing and/or crossing with a plant from the F1 population to obtain a further generation;
c) selecting a plant that comprises the modified YLS9 gene homozygously, the modified HsfA2 gene homozygously, or both modified genes homozygously as a resistant plant.

30. A method for producing hybrid seed resistant to a Begomovirus comprising the steps of
crossing a first parent plant with a second parent plant, wherein both parent plants are homozygous for the modified YLS9 gene of claim 1 and/or the modified HsfA2 of claim 6, and
harvesting the hybrid seed.

31. The method of claim 27 or 28, wherein the Begomovirus is ToLCNDV and/or ToLCPMV.

32. The hybrid seed produced by the method of claim 30.

33. A plant grown from the hybrid seed of claim 31.

34. The method of claim 29, wherein the Begomovirus is ToLCNDV and/or ToLCPMV.

35. The method of claim 30, wherein the Begomovirus is ToLCNDV and/or ToLCPMV.
